(19) United States
(12) Patent Application Publication
(10) Pub. No.: US 2004/0009231 A1
(43) Pub. Date: Jan. 15, 2004

(54) hGH (human growth hormone) formulations for pulmonary administration

(75) Inventors: Blair Jackson, Quincy, MA (US); Lloyd Johnston, Belmont, MA (US); Charles D. Blizzard, Westwood, MA (US); Jeffrey Mintzes, Monsey, NY (US)

Correspondence Address: Elmorc Craig, P.C., 209 Main Street, No. Chelmsford, MA, 01863, US

(73) Assignee: Advanced Inhalation Research, Inc., Cambridge, MA

(21) Appl. No.: 10/394,401

(22) Filed: Mar. 19, 2003

Related U.S. Application Data
(60) Provisional application No. 60/366,488, filed on Mar. 20, 2002.

(51) Int. Cl. A61K 38/27; A61K 9/14

(52) U.S. Cl. 424/489; 514/12

(57) ABSTRACT

This invention relates to the administration of proteins by absorption from the lungs. In particular, it is concerned with providing therapeutic doses of human growth hormone to the bloodstream without irritating or otherwise damaging lung tissue. This invention also relates to the methods of delivery of human growth hormone to the pulmonary system.

### RELATED APPLICATION

This application claims the benefit of U.S. Provisional Application No. 60/366,488, filed Mar. 20, 2002. This application is related to PCT Application entitled “hGH (Human Growth Hormone) Formulations for Pulmonary Administration”, under Attorney Docket No. 2685.2040003 and PCT Application entitled “Method for Administration of Growth Hormone Via Pulmonary Delivery”, filed concurrently herewith under Attorney Docket No. 2685.2040005. The entire teachings of the above applications are incorporated herein by reference.

### BACKGROUND OF THE INVENTION

Aerosols for the delivery of therapeutic agents to the respiratory tract have been described, for example, Adjei, A. and Garren, J. Pharm. Res., 7:565-569 (1990); and Zanen, P. and Lamm, J.-W. J., Int. J. Pharm., 114:111-115 (1995). The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. Gonda, I., “Aerosols for delivery of therapeutic and diagnostic agents to the respiratory tract,” in Critical Reviews in Therapeutic Drug Carrier Systems, 6:273-313 (1990). The deep lung or alveoli are the primary target of inhaled therapeutic aerosols for systemic drug delivery.

Inhaled aerosols have been used for the treatment of local lung disorders including asthma and cystic fibrosis (Anderson, Am. Rev. Respir. Dis., 140:1317-1324 (1989)) and have potential for the systemic delivery of peptides and proteins as well (Patton and Platz, Advanced Drug Delivery Reviews, 8:179-196 (1992)).

There are reported examples of growth hormone formulations with stabilizing excipients such as mannitol, glycine, arginine and lactose. Some current formulations of hGH lose activity due to formation of dimer and higher order aggregates (macro range) during formulation processing as well as during storage and reconstitution. Other chemical changes, such as deamidation and oxidation may also occur upon storage. However, it is not feasible to foresee a standard formulation for all the proteins, and the choice of the best formulation requires a remarkable selection work.

However, pulmonary drug delivery strategies present many difficulties, in particular for the delivery of macromolecules such as hGH; these include protein denaturation during aerosolization, excessive loss of inhaled drug in the oropharyngeal cavity (often exceeding 80%), poor control over the site of deposition, lack of reproducibility of therapeutic results owing to variations in breathing patterns, the frequent too-rapid absorption of drug potentially resulting in local toxic effects, and phagocytosis by lung macrophages.

In addition, many of the devices currently available for inhalation therapy are associated with drug losses. Considerable attention has been devoted to the design of therapeutic aerosol inhalers to improve the efficiency of inhalation therapies. Timsina et al., Int. J. Pharm., 101: 1-13 (1995) and Tansey, I. P., Spray Technol. Market, 4:26-29 (1994). Attention has also been given to the design of dry powder aerosol surface texture, regarding particularly the need to avoid particle aggregation, a phenomenon which considerably diminishes the efficiency of inhalation therapies. French, D. L., Edwards, D. A. and Niven, R. W., J. Aerosol Sci., 27:769-783 (1996).

In order that materials like hGH be provided to health care personnel and patients, these materials must be prepared as pharmaceutical compositions. Such compositions must maintain activity for appropriate periods of time, must be acceptable in their own right to easy and rapid administration to humans, and must be readily manufacturable. In many cases, pharmaceutical formulations are provided in frozen or in lyophilized form. The frozen or lyophilized composition is often used to maintain biochemical integrity and the bioactivity of the medicinal agent contained in the compositions under a wide variety of storage conditions, as it is recognized by those skilled in the art that lyophilized preparations often maintain activity better than their liquid counterparts. However, such frozen or lyophilized preparations must be thawed or reconstituted prior to use by the addition of suitable pharmaceutically acceptable diluent(s), such as sterile water for injection or sterile physiological saline solution, and the like.

Alternatively, the compositions can be provided in a dry powder formulation (DPF). DPF's are gaining increased interest as aerosol formulations for pulmonary delivery. Damms, B. and W. Bains, Nature Biotechnology (1996); Kobayashi, S., et al., Pharm. Res., 13(1):80-83 (1996); and Timsina, M. et al., Int. J. Pharm., 101:1-13 (1994). Dry powder aerosols for inhalation therapy are generally produced with mean geometric diameters primarily in the range of less than 5 μm. Ganderton, D., J. Biopharmaceutical Sciences, 3:101-105 (1992) and Gonda, I., “Physico-Chemical Principles in Aerosol Delivery,” Topics in Pharmaceutical Sciences (1991), Crommelin, D. J. and K. K. Midha, Eds., Medpharm Scientific Publishers, Stuttgart, pp. 95-115, 1992. Large “carrier” particles (containing no drug) have been co-delivered with therapeutic aerosols to aid in achieving efficient aerosolization among other possible benefits. French, D. L., Edwards, D. A. and Niven, R. W., J Aerosol Sci., 27:769-783 (1996). Formulations and methods of administering them are also described in U.S. application Ser. No. 09/591,307 (High Efficient Delivery of a Large Therapeutic Mass Aerosol) and Ser. No. 09/878,146 (Highly Efficient Delivery of a Large Therapeutic Mass Aerosol), filed, respectively on Jun. 9, 2000 and Jun. 8, 2001; the entire contents of both is incorporated by reference herein.

Among the disadvantages of DPF's is that powders of fine particulates usually have poor flowability and aerosolization properties, leading to relatively low respirable fractions of aerosol, which are the fractions of inhaled aerosol that deposit in the lungs, resulting in deposition of the aerosol in the mouth and throat. Gonda, I., in Topics in Pharmaceutical Sciences, (1991), D. Crommelin and K. Midha, Editors, Stuttgart: Medpharm Scientific Publishers, pp. 95-117 (1992). Poor flowability and aerosolization properties are typically caused by particulate aggregation, due to particle-particle interactions, such as hydrophobic, electrostatic, and capillary interactions. Some improvements in DPF's have been made. For example, dry powder formulations (“DPFs”) with large particle size have been shown to possess improved flowability characteristics, such as less aggregation (Edwards, et al., Science 276:1868-1871 (1997)), easier aerosolization, and potentially less phagocytosis. Rudt, S. and R. H. Muller, J., Controlled Release, 22:263-272 (1992); Tabata, Y. and Y. Ikada, J. Biomed. Mater. Res., 22:837-858 (1988). An effective dry-powder inhalation therapy for both short and long term release of therapeutics, either for local or systemic delivery, requires a method to deliver a DPF to the lungs efficiently, and at therapeutic levels, without requiring excessive energy input.

### SUMMARY OF THE INVENTION

This invention relates to the administration of proteins by absorption from the lungs. In particular, it is concerned with providing therapeutic doses of human growth hormone to the bloodstream without irritating or otherwise damaging lung tissue. This invention also relates to the methods of delivery of human growth hormone to the pulmonary system.

The pharmaceutical formulations of the invention comprise particles, by weight, approximately 75% to about 100% hGH and approximately 3% to about 20% sodium phosphate, e.g., provided by using sodium phosphate monohydrate, are disclosed. Optionally, the particles further comprise, by weight, approximately 5% to about 18% 1,2-dipalmitoyl-sn-glycero-3-phospatidylcholine (DPPC). In one embodiment the particles are contained in a receptacle that comprises a mass of from between about 1.0 mg and about 25 mg of hGH. In a further embodiment, the particles have a tap density less than about 0.4 g/cm³, a median geometric diameter of from between about 1 micrometers and about 30 micrometers, for example, but not limited to, between about 5 micrometers and 10 micrometers, and an aerodynamic diameter of from about 1 micrometer to about 5 micrometers.

The invention also relates to methods for treating a human patient in need of hGH comprising administering to the respiratory tract of a patient in need of treatment, in a single, breath actuated step an effective amount of particles comprising, by weight, approximately 75% to about 100% hGH and approximately 3% to about 20% sodium phosphate. In yet another embodiment of the method, the particles further comprise, by weight, approximately 5% to about 18% DPPC. In a further embodiment, the particles have a tap density less than about 0.4 g/cm³, a median geometric diameter of from between about 1 micrometers and about 30 micrometers, for example, but not limited to, between about 5 micrometers and 10 micrometers, and an aerodynamic diameter of from about 1 micrometer to about 5 micrometers.

Methods of delivering an effective amount of hGH to the pulmonary system, comprising a) providing a mass of particles comprising, by weight, approximately 75% to about 100% hGH and approximately 3% to about 20% sodium phosphate; and b) administering via simultaneous dispersion and inhalation the particles, from a receptacle having the mass of the particles, to a human subject's respiratory tract. In yet another embodiment of the method, the particles further comprise, by weight, approximately 5% to about 18% DPPC. In a further embodiment, the particles have a tap density less than about 0.4 g/cm³, a median geometric diameter of from between about 1 micrometers and about 30 micrometers, for example, but not limited to, between about 5 micrometers and 10 micrometers, and an aerodynamic diameter of from about 1 micrometer to about 5 micrometers.

The invention has numerous advantages. For example, particles suitable for inhalation can be designed to possess a controllable release profile. Rapid release is preferred. This rapid release profile provides for abbreviated residence of the administered bioactive agent, in particular hGH, in the lung and decreases the amount of time in which therapeutic levels of the agent are present in the local environment or systemic circulation.

The rapid release of agent provides a desirable alternative to injection therapy currently used for many therapeutic, diagnostic and prophylactic agents requiring rapid release of the agent, such as hGH. In addition, the invention provides a method of delivery to the pulmonary system wherein the high initial release of agent typically seen in inhalation therapy is boosted, giving very high initial release. Consequently, patient compliance and comfort can be increased by not only reducing frequency of dosing, but by providing a therapy that is more amenable to patients. Moreover, particle formulated using hGH and non-phospholipidic excipient, such as sodium phosphate monohydrate have the further advantages of making the particles easier to manufacture (one less excipient/ingredient to dispense—thus requires one less mixing step), less expensive to manufacture (phospholipids such as DPPC are expensive), easier to increase the scale of particle production (the presence of phospholipids creates solubility limitations that requires heating of the solutions during mixing) and higher hGH levels/concentrations in the particle formulations.

This dry powder delivery system allows for efficient dose delivery from a small, convenient and inexpensive delivery device. In addition, the simple and convenient inhaler together with the room temperature stable powder may offer an attractive replacement for currently available injectable formulations. This system has the potential to help achieve improved therapeutic effects of hGH by increasing the willingness of patients to comply with hGH therapy.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIGS. 1A-1C are graphs showing the plot of the hGH concentrations in the blood as a function of time of 12 individuals to whom two dry powder inhaled formulations which are embodiments of the instant invention were administered.

FIG. 1A shows the plot of hGH concentrations in the blood as a function of time for one of the hGH formulations (80% hGH, 14% DPPC and 6% sodium phosphate, by weight).

FIG. 1B shows the plot of hGH concentrations in the blood as a function of time for another of the hGH formulations (93% hGH and 7% sodium phosphate, by weight).

FIG. 1C shows the plot of hGH concentrations in the blood as a function of time for subcutaneously administered doses of hGH.

FIGS. 2A-2C are schematics contrasting the summary of pharmokinetic (Pk) parameters for two dry powder inhaled formulations of hGH, which are embodiments of the instant invention, and the subcutaneous dosing of hGH.

FIG. 2A depicts the Pk profile for one of the formulations (80% hGH, 14% DPPC and 6% sodium phosphate, by weight).

FIG. 2B depicts the Pk profile for another of the formulations (93% hGH and 7% sodium phosphate, by weight).

FIG. 2C depicts the Pk profile for subcutaneously administered hGH.

FIGS. 3A-3B are schematics contrasting the individual C max and AUC values for 12 individuals to whom two dry powder inhaled formulations, which are embodiments of the instant invention, were administered and the subcutaneous dosing of hGH of these individuals.

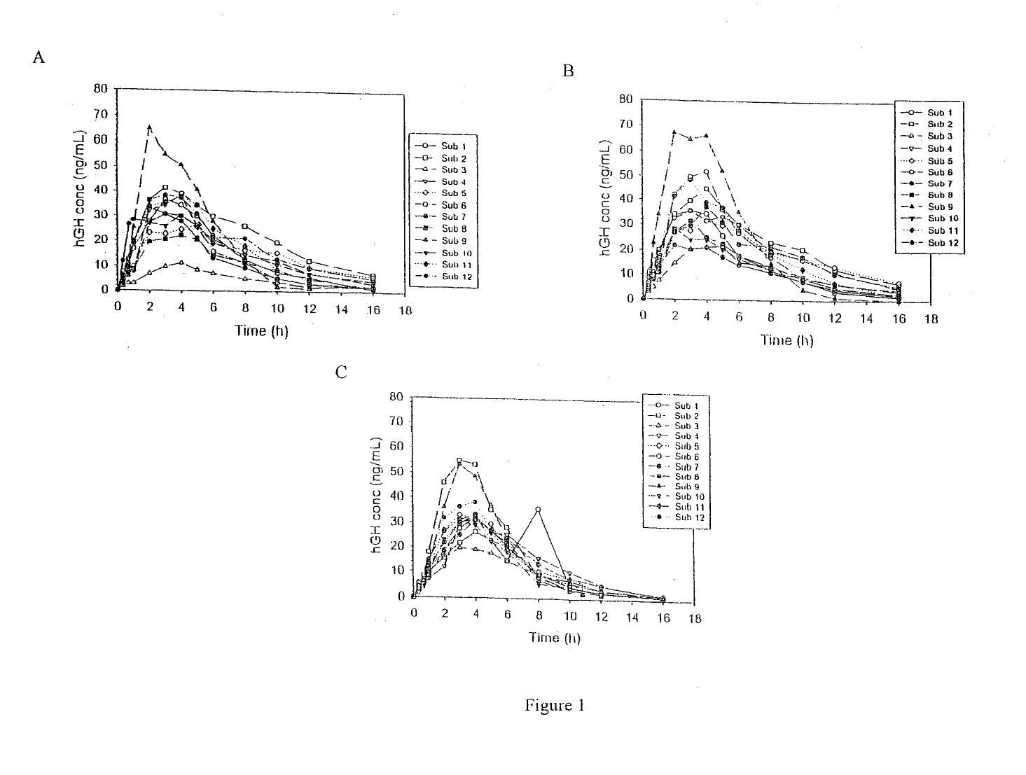

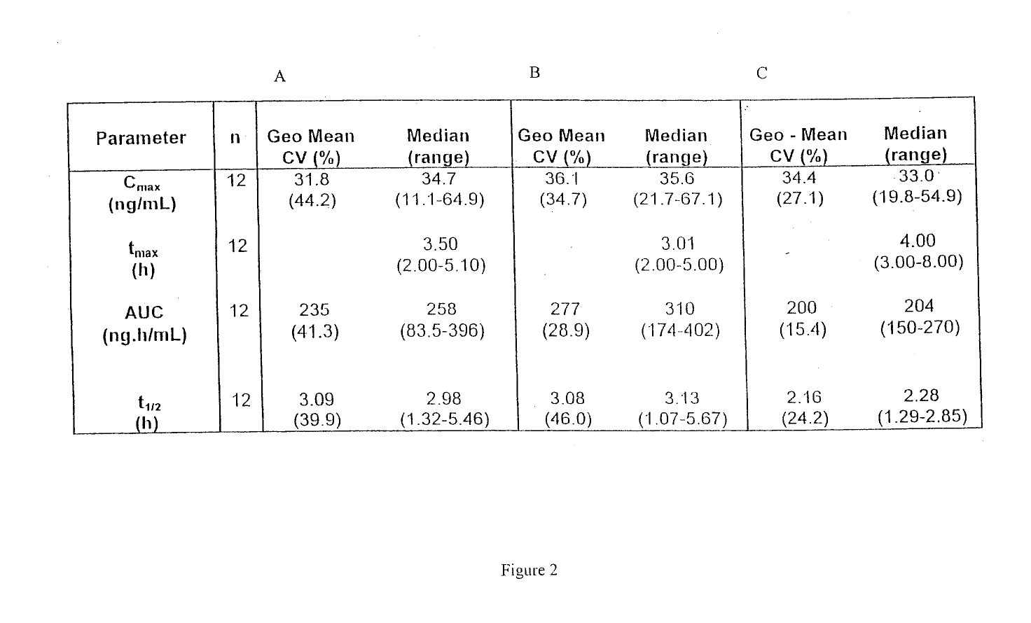

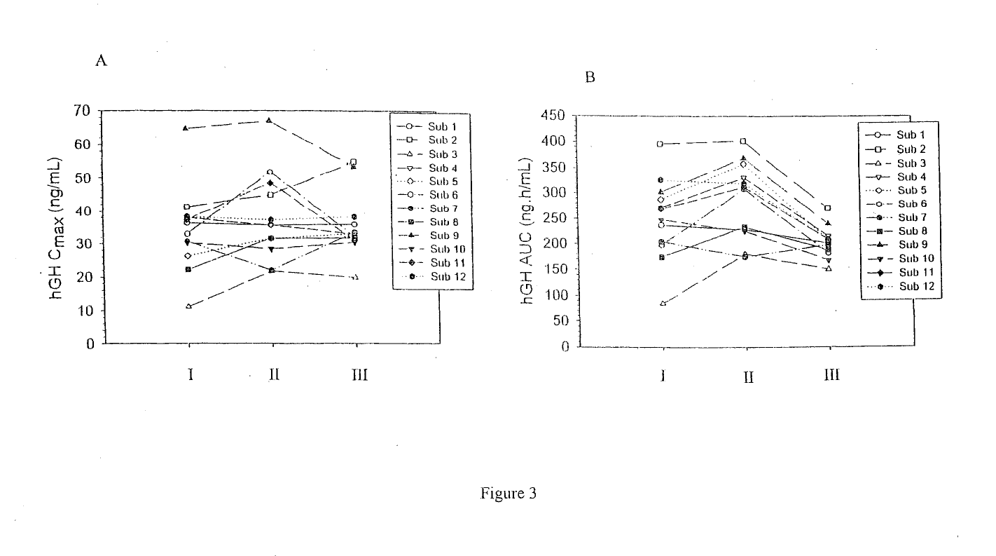

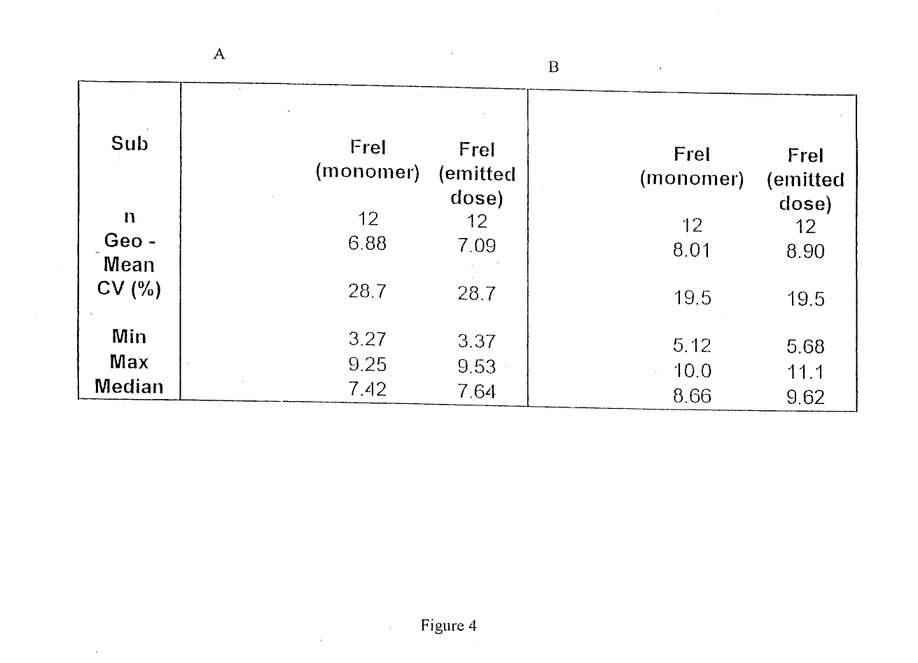

Label 3AI corresponds to the Cₘₐₓ values for one of the formulations (80% hGH, 14% DPPC and 6% sodium phosphate, by weight).

Label 3AII corresponds to the Cₘₐₓ values for another of the formulations (93% hGH and 7% sodium phosphate, by weight).

Label 3AIII corresponds to the Cₘₐₓ values for subcutaneously administered hGH.

Label 3BI corresponds to the AUC values for one of the formulations (80% hGH, 14% DPPC and 6% sodium phosphate, by weight).

Label 3BII corresponds to the AUC values for another of the formulations (93% hGH and 7% sodium phosphate, by weight).

Label 3BIII corresponds to the AUC values for subcutaneously administered hGH.

FIGS. 4A-4B are charts summarizing the relative bioavailability for two dry powder inhaled formulations of hGH, which are embodiments of the instant invention, relative to the subcutaneous dosing of hGH.

FIG. 4A depicts the relative bioavailability for one of the formulations (80% hGH, 14% DPPC and 6% sodium phosphate, by weight).

FIG. 4B depicts the relative bioavailability for another of the formulations (93% hGH and 7% sodium phosphate, by weight).

### DETAILED DESCRIPTION OF THE INVENTION

The features and other details of the invention, either as steps of the invention or as combination of parts of the invention, will now be more particularly described with reference to the accompanying drawings and pointed out in the claims. It will be understood that the particular embodiments of the invention are shown by way of illustration and not as limitations of the invention. The principle feature of this invention may be employed in various embodiments without departing from the scope of the invention.

The invention relates to particles that include a growth hormone and methods of producing and delivering the particles to the pulmonary system. Growth hormones or factors are polypeptides that induce the proliferation or enlargement of target cells. Growth hormone is also a key hormone involved in the regulation of somatic growth and in the regulation of metabolism of proteins, carbohydrates and lipids. The organ systems affected include the skeleton, connective tissue, muscles and viscera such as liver, intestine and kidneys. As used herein, the term “growth hormone” includes homologs, analogs, allelic-variants, mutants, fragments and complementary nucleic acid sequences of the native molecule. These variants may exhibit enhanced levels of the normal biological activity of the native molecules or may, on the contrary, act antagonistically towards the native molecule. Alternatively, variants are selected for improved characteristics such as stability to oxidation, extended biological half-life, and the like. Such variants as are known or will be developed in the future are suitable for use herein. For example, N-terminal methionyl human growth hormone (somatrem) is a common variant produced in recombinant cell culture wherein a methionine residue not found in the native analogue is covalently bound to the normal N-terminal amino acid residue.

Particularly preferred in the compositions and methods of the invention is human growth hormone (hGH). Human growth hormone is secreted in the human pituitary and its major effect is to promote growth. In its mature form it consists of 191 amino acids, has a molecular weight of about 22,000 kDa and thus is more than three times as large as insulin. This hormone is a linear polypeptide containing two intrachain disulfide bridges. Until the advent of recombinant DNA technology, hGH could be obtained only by laborious extraction from a limited source: the pituitary glands of human cadavers. The consequent scarcity of substance limited its application to treatment of hypopituitary dwarfism. hGH has also been proposed to be effective in the treatment of burns, wound healing, dystrophy, bone knitting, diffuse gastric bleeding and pseudarthrosis. hGH can be produced in a recombinant host cell, in quantities which would be adequate to treat hypopituitary dwarfism and the other conditions for which it is effective.

The particles of the invention are useful for delivery of hGH to the pulmonary system, in particular to the deep lung. The particles are in the form of a dry powder and are characterized by a fine particle fraction (FPF), geometric and aerodynamic dimensions and by other properties, as further described herein. As used herein, irrespective of the weight percent for the formulations as described herein, the particles are understood by those of skill in the art to have a moisture and/or residual solvent content. Typically, the moisture and residual solvent content of the particles will be below 10 weight percent (wt %), or below 7 wt %, or below 5 wt %.

The particles disclosed herein include natural, synthetic (i.e. produced on the basis of recombinant DNA technology) hGH or combinations of natural and synthetic hGH. In one embodiment, the particles of the invention are used to treat adult and pediatric Growth Hormone Deficient (GHD) patients. In another embodiment, the particles are used to treat patients suffering from non-growth hormone deficiency disorders treatable with hGH which include: Turner Syndrome in patients whose epiphyses are not closed; Non-Growth Hormone Deficient Short Stature (NGHDSS); Small for Gestational Age (SGA); SHOX deficiency; achondroplasia; Prader-Willi Syndrome; chronic renal insufficiency; AIDS; and, for any other indication of hGH.

The particles of the invention include at least about 75 percent by weight hGH, preferably at least 90 weight percent hGH. Particularly preferred are particles that include at least 90 weight percent hGH, for instance, at least 93 weight percent hGH. In one embodiment, the particles include as much as 93% hGH by weight. In another embodiment, the particles include as much as 93.5% hGH by weight.

Pharmaceutical formulations which are suitable for pulmonary delivery comprise particles that include, by weight, approximately 75% to about 100% hGH and approximately 3% to about 20% sodium phosphate monohydrate. In another embodiment of the formulation, the particles further comprise, by weight, approximately 5% to about 18% 1,2-dipalmitoyl-sn-glycero-3-phospatidylcholine (DPPC). The particles have a tap density less than about 0.4 g/cm³, a median geometric diameter of from between about 5 micrometers and about 30 micrometers, and an aerodynamic diameter of from about 1 micrometer to about 5 micrometers.

The particles of the invention also include a buffer salt, such as sodium phosphate, ammonium bicarbonate and others. Sodium phosphate is preferred. Sodium phosphate generally is provided in the form, but not limited to, sodium phosphate monohydrate or sodium phosphate dibasic. Combinations of buffer salts also can be employed. The amount of buffer salt(s), e.g., sodium phosphate present in the particles of the invention generally is less than 20 weight percent. For example, the amount of sodium phosphate is less than 15 weight percent, and even less than 10 weight percent.

In one embodiment of the invention, the particles consist essentially of growth hormone, e.g., hGH and buffer salt(s).

In other embodiments the particles include one or more additional components. Generally, the amount of the additional component(s) is less than 50 weight percent, preferably less than 30 weight percent and most preferably less than 20 weight percent. For example, particles include, in addition to the growth hormone and buffer salt(s), one or more phospholipids. Specific examples of phospholipids include but are not limited to phosphatidylcholines dipalmitoyl phosphatidylcholine (DPPC), dipalmitoyl phosphatidylethanolamine (DPPE), distearoyl phosphatidylcholine (DSPC), dipalmitoyl phosphatidyl glycerol (DPPG) or any combination thereof.

The phospholipids or combinations thereof are selected to impart controlled release properties to the highly dispersible particles. The phase transition temperature of a specific phospholipid can be below, around or above the physiological body temperature of a patient, such that the phase transition temperatures range from 30° C. to 50° C., (e.g., within ±10° C. of the normal body temperature of patient). By selecting phospholipids or combinations of phospholipids according to their phase transition temperature, the particles are tailored to have controlled release properties. For example, rapid release is obtained by including in the particles phospholipids having low transition temperatures. Particles having controlled release properties and methods of modulating release of a biologically active agent are described in U.S. application Ser. No. 09/792,869 entitled “Modulation of Release from Dry Powder Formulations”, filed on Feb. 23, 2001, which is a continuation-in-part of U.S. application Ser. No. 09/644,736 entitled “Modulation of Release from Dry Powder Formulations”, filed on Aug. 23, 2000, both of which claim the benefit of U.S. Provisional Patent Application No. 60/150,742 entitled “Modulation of Release From Dry Powder Formulations by Controlling Matrix Transition”, filed on Aug. 25, 1999. The contents of all three applications are incorporated herein by reference in their entirety.

Other suitable components that can be used in the particles of the invention include, but are not limited to, amino acids, in particular hydrophobic amino acids, e.g., leucine. Methods of forming and delivering particles which include an amino acid are described in U.S. application Ser. No. 09/644,320, filed on Aug. 23, 2000, entitled “Use of Simple Amino Acids to Form Porous Particles”, which is a continuation-in-part of U.S. patent application Ser. No. 09/382,959, filed on Aug. 25, 1999, entitled “Use of Simple Amino Acids to Form Porous Particles During Spray Drying”. The entire teachings of both applications is incorporated herein by reference.

In a further embodiment, the particles can also include other materials such as, for example, buffer salts, dextran, polysaccharides, lactose, sucrose, trehalose, cyclodextrins, proteins, peptides, polypeptides, fatty acids, fatty acid esters, inorganic compounds, phosphates, salts, sugars, polymers and surfactants.

In one embodiment of the invention, the particles comprise polymers. The use of polymers can further prolong release. Biocompatible or biodegradable polymers are preferred. Such polymers are described, for example, in U.S. Pat. No. 5,874,064, issued on Feb. 23, 1999 to Edwards et al., the teachings of which are incorporated herein by reference in their entirety.

In another embodiment, the particles include a surfactant. As used herein, the term “surfactant” refers to any agent which preferentially absorbs to an interface between two immiscible phases, such as between a water/organic interface, a water/air interface, or organic solvent/air interface. Surfactants generally possess a hydrophilic moiety and a lipophilic moiety, such that, upon absorbing to the microparticles, they tend to present moieties to the external environment that do not attract similarly-coated molecules, thus reducing hGH molecule agglomeration. Surfactants may also promote absorption of a therapeutic or diagnostic agent and increase bioavailability of the agent.

Suitable surfactants which can be employed in fabricating the particles of the invention include but are not limited to Tween-20; Tween-80; hexadecanol; fatty alcohols such as polyethylene glycol (PEG); polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); and tyloxapol. Tween-20 and/or Tween-80 can range from about 0.01 weight percent to about 11.2 weight percent, for example, but not limited to, about 0.2 weight percent to about 2.8 weight percent. The addition of Tween may increase the readily extractable fraction. The readily extractable fraction refers to the % of agent, e.g., hGH, that is released from the particles.

The particles of the invention are suitable for delivery to the respiratory system, and are especially useful to deliver a growth hormone to the deep lung. The “respiratory system”, as defined herein, encompasses the upper airways, including the oropharynx and larynx, followed by the lower airways, which include the trachea followed by bifurcations into the bronchi and bronchioli (e.g., terminal and respiratory). The upper and lower airways are called the conducting airways. The terminal bronchioli then divide into respiratory bronchioli which then lead to the ultimate respiratory zone, namely, the alveoli, or deep lung. The deep lung, or alveoli, are typically the desired target of inhaled therapeutic formulations for systemic drug delivery. The particles of the invention are inhaled/inspired, administered to the mouth/upper respiratory tract of a subject, e.g., human or animal in need thereof. “Pulmonary pH range”, as that term is used herein, refers to the pH range which is encountered in the lung of a patient. Typically, in humans, this range of pH is from about 6.4 to about 7.0, such as from 6.4 to about 6.7. pH values of the airway lining fluid (ALF) have been reported in “Comparative Biology of the Normal Lung”, CRC Press, (1991) by R. A. Parent and range from 6.44 to 6.74.

The particles are administered as part of a pharmaceutical formulation or in combination with other therapies be they oral, pulmonary, injection or other mode of administration. As described herein, particularly useful pulmonary formulations are spray dried particles having physical characteristics which favor target lung deposition and are formulated to optimize release and bioavailability profiles.

Gravimetric analysis, using Cascade impactors, is a method of measuring the size distribution of airborne particles. The Andersen Cascade Impactor (ACI) is an eight-stage impactor that separates aerosols into nine distinct fractions based on aerodynamic size. The size cutoffs of each stage are dependent upon the flow rate at which the ACI is operated. For example, an eight-stage ACI (ACI-8) configuration can consist of 20 μm pore (stages −1, 0 and 1) and 150 μm pore (stages 2 through 6) stainless steel screens. The stages of the ACI can also be “wetted” by saturating the screens in methanol. Preferably the ACI is calibrated at 60 L/min. In one embodiment, an ACI-8 is used for particle optimization. In another embodiment, a two-stage collapsed ACI (ACI-2) is used for particle optimization. The two-stage collapsed ACI consists of only the top two stages of the eight-stage ACI and allows for the collection of two separate powder fractions. At each stage an aerosol stream passes through the nozzles and impinges upon the surface. Particles in the aerosol stream with a large enough inertia will impact upon the plate. Smaller particles that do not have enough inertia to impact on the plate will remain in the aerosol stream and be carried to the next stage.

The ACI-2 is calibrated so that the fraction of powder that is collected on a first stage is referred to as fine particle fraction (FPF) (5.6). This FPF corresponds to the percent (%) of particles that have an aerodynamic diameter of less than 5.6 μm. The fraction of powder that passed the first stage of the ACI and is deposited on the collection filter is referred to as FPF(3.4). This corresponds to the % of particles having an aerodynamic diameter of less than 3.4 μm.

The FPF (5.6) fraction has been demonstrated to correlate to the fraction of the powder that is deposited in the lungs of the patient, while the FPF(3.4) has been demonstrated to correlate to the fraction of the powder that reaches the deep lung of a patient.

The FPF of at least 50% of the particles of the invention is less than about 5.6 μm. For example, the FPF of at least 65% of the particles is less than 5.6 μm, or the FPF of at least 80% of the particles is less than 5.6 μm.

In a further embodiment, a three-stage ACI (ACI-3) is used for particle optimization. The ACI-3 consists of only the top three stages of the eight-stage ACI and allows for the collection of three separate powder fractions. For example, the ACI-3 configuration can consist of 20 μm pore (stages −1 and 1) and 150 μm pore (stage 2) stainless steel screens which can be “wet” (i.e., saturated with methanol). The fraction of the powder that passes the final stage is referred to as FPF(3.3).

Another method for measuring the size distribution of airborne particles is the multi-stage liquid impinger (MSLI). The MSLI operates on the same principles as the Anderson Cascade Impactor (ACI), but instead of eight stages there are five in the MSLI. Additionally, instead of each stage consisting of a solid plate, each MSLI stage consists of a methanol-wetted glass frit. The wetted stage is used to prevent bouncing and re-entrainment, which can occur using the ACI. The MSLI is used to provide an indication of the flow rate dependence of the powder. This is accomplished by operating the MSLI at 30, 60, and 90 L/min and measuring the fraction of the powder collected on stage 1 and the collection filter. If the fractions on each stage remain relatively constant across the different flow rates then the powder is considered to be approaching flow rate independence.

The particles of the invention have a tap density of less than about 0.4 g/cm³. Particles which have a tap density of less than about 0.4 g/cm³ are referred to herein as “aerodynamically light particles”. For example, the particles have a tap density less than about 0.3 g/cm³, or a tap density less than about 0.2 g/cm³, or a tap density less than about 0.1 g/cm³. Tap density is measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10^{th} Supplement, 4950-4951, 1999. Features which contribute to low tap density include irregular surface texture and porous structure.

The particles of the invention have a preferred size, e.g., a volume median geometric diameter (VMGD) of at least about 1 micron (μm). In one embodiment, the VMGD is from about 1 μm to 30 μm, or any subrange encompassed by about 1 μm to 30 μm, for example, but not limited to, from about 5 μm to about 30 μm, or from about 10 μm to 30 μm. For example, the particles have a VMGD ranging from about 1 μm to 10 μm, or from about 3 μm to 7 μm, or from about 5 μm to 15 μm or from about 9 μm to about 30 μm. The particles have a median diameter, mass median diameter (MMD), a mass median envelope diameter (MMED) or a mass median geometric diameter (MMGD) of at least 1 μm, for example, 5 μm or near to or greater than about 10 μm. For example, the particles have a MMGD greater than about 1 μm and ranging to about 30 μm, or any subrange encompassed by about 1 μm to 30 μm, for example, but not limited to, from about 5 μm to 30 μm or from about 10 μm to about 30 μm.

The geometric diameter can be measured using a RODOS dry powder disperser in conjunction with a HELOS laser diffractometer. Powder is introduced into the RODOS inlet and aerosolized by shear forces generated by a compressed air stream regulated from about 0.5 bar to about 4 bar. The aerosol cloud is subsequently drawn into the measuring zone of the HELOS, where it scatters light from a laser beam and produces a Fraunhofer diffraction pattern used to infer the particle size distribution. Other instruments for measuring particle diameter are well known in the art. The diameter of particles in a sample will range depending upon factors such as particle composition and methods of synthesis. The distribution of size of particles in a sample can be selected to permit optimal deposition within targeted sites within the respiratory tract.

The particles of the invention have “mass median aerodynamic diameter” (MMAD), also referred to herein as “aerodynamic diameter”, between about 1 μm and about 5 μm or any subrange encompassed between about 1 μm and about 5 μm. For example, but not limited to, the MMAD is between about 1 μm and about 3 μm, or the MMAD is between about 3 μm and about 5 μm.

The particles administered are highly dispersible. As used herein, the phrase “highly dispersible” particles or powders refers to particles or powders which can be dispersed by a RODOS dry powder disperser (or equivalent technique) such that at about 1 Bar, particles of the dry powder emit from the RODOS orifice with geometric diameters, as measured by a HELOS or other laser diffraction system, that are less than about 2 times the geometric particle size as measured at 4 bar, and preferably less than about 1.5 times the geometric particle size as measured at 4 bar. Some highly dispersible powders display ratios of less than 2 to 1, and even less than 1.5 to 1, when comparing 0.5 and 2 bar values. Highly dispersible powders have a low tendency to agglomerate, aggregate or clump together and/or, if agglomerated, aggregated or clumped together, are easily dispersed or de-agglomerated as they emit from an inhaler and are breathed in by the subject. Typically, the highly dispersible particles suitable in the methods of the invention display very low aggregation compared to standard micronized powders which have similar aerodynamic diameters and which are suitable for delivery to the pulmonary system. Properties that enhance dispersibility include, for example, particle charge, surface roughness, surface chemistry and relatively large geometric diameters. Because the attractive forces between particles of a powder varies (for constant powder mass) inversely with the square of the geometric diameter and the shear force seen by a particle increases with the square of the geometric diameter, the ease of dispersibility of a powder is on the order of the inverse of the geometric diameter raised to the fourth power. The increased particle size diminishes interparticle adhesion forces. (Visser, J., Powder Technology, 58:1-10 (1989)). Thus, large particle size, all other things equivalent, increases efficiency of aerosolization to the lungs for particles of low envelope mass density. Increased surface irregularities, and roughness also can enhance particle dispersibility. Those skilled in the art are able to measure the irregularities and roughness. Surface roughness can be expressed, for example by rugosity.

Experimentally, aerodynamic diameter can be measured using an AeroDisperser/Aerosizer. The sample powder was aerosolized by an inlet air stream at 1 psi in the AeroDisperser and then accelerated to sonic velocity into the Aerosizer. The Aerosizer measures the time taken for each particle to pass between two fixed laser beams, which is dependent on the particle's inertia. The time of flight (TOF) measurements were subsequently converted into aerodynamic diameters using Stokes law. Additionally, the aerodynamic diameter can be determined by employing a gravitational settling method, whereby the time for an ensemble of particles to settle a certain distance is used to infer directly the aerodynamic diameter of the particles. The MSLI also provides an indirect method for measuring the mass median aerodynamic diameter.

The aerodynamic diameter, dₐₑᵣ, can be calculated from the equation:

dₐₑᵣ=d_{g}√ρtap

where d_{g} is the geometric diameter, for example the MMGD and ρ is the powder density.

Particles which have a tap density less than about 0.4 g/cm³, median diameters of at least about 1 μm, for example, at least about 5 μm, and an aerodynamic diameter of between about 1 μm and about 5 μm, preferably between about 1 μm and about 3 μm, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

In comparison to smaller particles the larger aerodynamically light particles, preferably having a VMGD of at least about 5 μm, are potentially more successfully avoid phagocytic engulfinent by alveolar macrophages and clearance from the lungs, due to size exclusion of the particles from the phagocytes' cytosolic space. Phagocytosis of particles by alveolar macrophages diminishes precipitously as particle diameter increases beyond about 3 μm. Kawaguchi, H., et al., Biomaterials 7: 61-66 (1986); Krenis, L. J. and Strauss, B., Proc. Soc. Exp. Med., 107: 748-750 (1961); and Rudt, S. and Muller, R. H., J. Contr. Rel., 22: 263-272 (1992). For particles of statistically isotropic shape, such as spheres with rough surfaces, the particle envelope volume is approximately equivalent to the volume of cytosolic space required within a macrophage for complete particle phagocytosis.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 μm are preferred for delivery to the central and upper airways. Particles having an aerodynamic diameter ranging from about 1 to about 3 μm are preferred for delivery to the deep lung.

Inertial impaction and gravitational settling of aerosols are predominant deposition mechanisms in the airways and acini of the lungs during normal breathing conditions. Edwards, D. A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

The low tap density particles have a small aerodynamic diameter in comparison to the actual envelope sphere diameter. As mentioned above, the aerodynamic diameter, dₐₑᵣ, is related to the envelope sphere diameter, d (Gonda, I., “Physico-chemical principles in aerosol delivery,” in Topics in Pharmaceutical Sciences 1991 (eds. D. J. A. Crommelin and K. K. Midha), pp. 95-117, Stuttgart: Medpharm Scientific Publishers, 1992)), by the formula:

dₐₑᵣ=d√ρ

where the envelope mass ρ is in units of g/cm³. Maximal deposition of monodispersed aerosol particles in the alveolar region of the human lung (˜60%) occurs for an aerodynamic diameter of approximately dₐₑᵣ=3 μm. Heyder, J. et al., J. Aerosol Sci., 17: 811-825 (1986). Due to their small envelope mass density, the actual diameter d of aerodynamically light particles comprising a monodisperse inhaled powder that will exhibit maximum deep-lung deposition is:

d=3/ρp μm (where √<g/cm³);

where d is always greater than 3 μm. For example, aerodynamically light particles that display an envelope mass density, ρ=0.1 g/cm³, will exhibit a maximum deposition for particles having envelope diameters as large as 9.5 μm. The increased particle size diminishes interparticle adhesion forces. Visser, J., Powder Technology, 58: 1-10. Thus, large particle size increases efficiency of aerosolization to the deep lung for particles of low envelope mass density, in addition to contributing to lower phagocytic losses.

The aerodynamic diameter can be calculated to provide for maximum deposition within the lungs of large particles which escape phagocytosis. Previously escaping phagocytosis was achieved by the use of very small particles with geometric diameters of less than about five microns in diameter, preferably between about one and about three microns. Selection of particles which have a larger geometric diameter or MMD, but which are sufficiently light (hence the characterization “aerodynamically light”), results in an equivalent delivery to the lungs, but the larger size particles are not phagocytosed. Improved delivery can be obtained by using particles with a rough or uneven surface relative to those with a smooth surface.

Suitable particles can be fabricated or separated, for example by filtration or centrifugation, to provide a particle sample with a preselected size distribution. For example, greater than about 30%, 50%, 70%, or 80% of the particles in a sample can have a diameter within a selected range of at least about 5 μm. The selected range within which a certain percentage of the particles must fall may be for example, between about 1 and about 30 μm, or between about 5 and about 30 μm, or between about 3 and about 11 μm, or between about 5 and about 15 μm. At least a portion of the particles have a diameter between about 1 and about 12 μm, or between about 3 and about 7 μm, or between about 4 and about 7 μm, or between about 4 and about 9 μm, or between about 5 and about 9 μm, or between about 5 and about 11 μm, or between about 7 and about 11 μm. Optionally, the particle sample also can be fabricated wherein at least about 90%, or optionally about 95% or about 99%, have a diameter within the selected range. The presence of the higher proportion of the aerodynamically light, larger diameter particles in the particle sample enhances the delivery of therapeutic or diagnostic agents incorporated therein to the deep lung. Large diameter particles generally mean particles having a median geometric diameter of at least about 5 μm.

This invention also relates to the preparation of growth hormone-containing particles. In one method of the invention particles that have the composition and properties described above are prepared by spray drying.

Specific examples of suitable equipment for spray drying is described in the exemplification section, below. Other equipment can be used, as known in the art.

Suitable spray-drying techniques are described, for example, by K. Masters in “Spray Drying Handbook”, John Wiley & Sons, New York, 1984.

A method for preparing a dry powder composition is provided. In this method, first and second components are prepared, one of which comprises an active agent. In such a method, the first component comprises an active agent dissolved in an aqueous solvent, and the second component comprises an excipient dissolved in an organic solvent. The first and second components are combined either directly or through a static mixer to form a combination. The first and second components are such that combining them causes degradation in one of the components. For example, the active agent in one component is incompatible with the other component. In this embodiment, the incompatible active agent (e.g., hGH) is added last. The combination is atomized to produce droplets that are dried to form dry particles. The atomizing step is performed immediately after the components are combined in the static mixer.

The aqueous solvent may further comprise ammonium bicarbonate. The use of ammonium bicarbonate in the spray drying solution is believed to increase the fine particle fraction of the particles. The amount of ammonium bicarbonate present in the aqueous solvent being spray dried is generally greater than about 6 g/L. For example, the amount of ammonium bicarbonate in the aqueous solvent is greater than about 10 g/L, for instance, greater than about 15 g/L, or greater than about 20 g/L.

The aqueous solvent is then mixed with an organic solvent which is then fed to the spray drier. Suitable organic solvents that can be present in the mixture being spray dried include, but are not limited to, alcohols for example, ethanol, methanol, propanol, isopropanol, butanols, and others. Other organic solvents include, but are not limited to, perfluorocarbons, dichloromethane, chloroform, ether, ethyl acetate, methyl tert-butyl ether and others. Aqueous solvents that can be present in the feed mixture include water and buffered solutions. Both organic and aqueous solvents can be present in the spray-drying mixture fed to the spray dryer. An ethanol water solvent is preferred with the ethanol:water ratio ranging from about 20:80 to about 10:90. The mixture can have an acidic or alkaline pH. Optionally, a pH buffer can be included. The pH can range from about 3 to about 10, or from about 6 to about 8.

A method for preparing a dry powder composition is provided, in which a first phase is prepared that comprises human growth hormone and sodium phosphate. The first phase may also comprise ammonium bicarbonate. A second phase is prepared that comprises ethanol. The first and second phases are combined to form a combination. The combination is atomized to produce droplets that are dried to form dry particles. In another aspect of such a method, the second phase further comprises 1,2-dipalmitoyl-sn-glycero-3-phosphatidylcholine (DPPC).

A method for preparing a dry powder composition is provided. In such a method, a first phase is prepared that comprises human growth hormone and sodium phosphate. The first phase may also comprise ammonium bicarbonate. A second phase is prepared that comprises ethanol. The first and second phases are combined in a static mixer to form a combination. The combination is atomized to produce droplets that are dried to form dry particles. In another aspect of such a method, the second phase further comprises 1,2-dipalmitoyl-sn-glycero-3-phosphatidylcholine (DPPC).

In one embodiment, the resulting dry particles consist of about 93 wt % human growth hormone and about 7 wt % sodium phosphate, for example, 93.5 wt % human growth hormone and 6.5 wt % sodium phosphate. In another embodiment, if DPPC is added to the second phase, the resulting particles consist of about 79 wt % human growth hormone, about 7 wt % sodium phosphate, and about 14 wt % DPPC.

An apparatus for preparing a dry powder composition is provided. The apparatus includes a static mixer (e.g., a static mixer as more fully described in U.S. Pat. No. 4,511,258, the entirety of which is incorporated herein by reference, or other suitable static mixers such as, but not limited to, model 1/4-21, made by Koflo Corporation.) having an inlet end and an outlet end. The static mixer is operative to combine an aqueous component with an organic component to form a combination. Means are provided for transporting the aqueous component and the organic component to the inlet end of the static mixer. An atomizer is in fluid communication with the outlet end of the static mixer to atomize the combination into droplets. The droplets are dried in a dryer to form dry particles. The atomizer can be a rotary atomizer. Such a rotary atomizer may be vaneless, or may contain a plurality of vanes. Alternatively, the atomizer can also be a two-fluid mixing nozzle. Such a two-fluid mixing nozzle may be an internal mixing nozzle or an external mixing nozzle, and may be a single-hole or six-hole two-fluid nozzle. The means for transporting the aqueous and organic components can be two separate pumps, or a single pump could be used. The aqueous and organic components can be transported to the static mixer at substantially the same rate. The apparatus can also include a geometric particle sizer that determines a geometric diameter of the dry particles, and an aerodynamic particle sizer that determines an aerodynamic diameter of the dry particles.

The total amount of solvent or solvents being employed in the mixture being spray dried generally is greater than 98 weight percent. The amount of solids (e.g., agent, phospholipid and other ingredients) present in the mixture being spray dried generally is greater than about 1 g/L. For example, the amount of solids in the mixture being spray dried is greater than about 3 g/L, for instance, at least 6 g/L, or at least about 12 g/L, or at least about 20 g/L.

In another embodiment, the total amount of solvent or solvents being employed in the mixture being spray dried generally is greater than 98 weight percent. The amount of solids (agent, phospholipid and other ingredients) present in the mixture being spray dried generally is less than about 2.0 weight percent. Preferably, the amount of solids in the mixture being spray dried ranges from about 0.1% to about 2% by weight.

The hGH solution combined either directly or through a static mixer is transferred to the 2-fluid nozzle atomizer at a flow rate of about 5 to 28 g/min (mass) and about 6 to 80 ml/min (volumetric). The hGH solution is transferred to the spray drier at a flow rate of 30 g/min and 31 ml/min. The 2-fluid nozzle disperses the liquid solution into a spray of fine droplets which come into contact with a heated drying air or heated drying gas (e.g., Nitrogen) under the following conditions:

The pressure within the nozzle is from about 10 psi to 100 psi; the heated air or gas has a feed rate of about 80 to 110 kg/hr and an atomization flow rate of about 13 to 67 g/min (mass) and a liquid feed of 10 to 50 ml/min (volumetric); a gas to liquid ratio from about 1:2 to 6:1; an inlet temperature from about 90° C. to 150° C.; an outlet temperature from about 40° C. to 70° C.; a baghouse outlet temperature from about 42° C. to 55° C. For example, the pressure within the nozzle is set at 75 psi; the heated gas feed rate is 110 kg/hr; and an atomizer gas flow rate of 46 g/min and a liquid feed rate of 25 ml/min; a gas to liquid ratio of 2: 1; an inlet temperature of 121° C.; an outlet temperature of 71° C.; and a baghouse temperature of 54° C.

The contact between the heated nitrogen and the liquid droplets causes the liquid to evaporate and porous particles to result. The resulting gas-solid stream is fed to the product filter, which retains the fine solid particles and allows that hot gas stream, containing the drying gas, evaporated water and ethanol, to pass. The formulation and spray drying parameters are manipulated to obtain particles with desirable physical and chemical characteristics. Other spray-drying techniques are well known to those skilled in the art. An example of a suitable spray dryer using 2-fluid atomization includes the Mobile Minor spray dryer, manufactured by Niro, Denmark. The hot gas can be, for example, air, nitrogen, carbon dioxide or argon.

As an example, particles of the present invention are made by the following process:

1. In vessel #1, dissolve hGH lyo-powder into 1.7 mM sodium phosphate buffer pH 7.4.

2. Carefully filter the contents of vessel #1 into vessel #2 at a flow rate of 100 ml/min.

3. Measure hGH concentration of the solution in vessel #2 using the UV/VIS spectrophotometer.

4. Use the total mass of hGH in vessel #2 as the basis for calculating the total solvents, total sodium phosphate and total ammonium bicarbonate needed in the solution. The final solution is comprised of 84%/16% WFI/ethyl alcohol (weight basis), 6 g/L solids comprised of 93%/7% hGH/sodium phosphate and 15 g/L ammonium bicarbonate.

5. Based on the calculation in step 4, add to a new vessel called #3 the remaining amount of WFI needed in the aqueous phase.

6. Based on the calculation in step 4, add the remaining amount of sodium phosphate needed to the WFI to vessel #3 and adjust the pH to 7.4 using 1.0 N sodium hydroxide.

7. Based on the calculation in step 4, add the necessary amount of ammonium bicarbonate to vessel #3.

8. Filter the contents of vessel #3 into vessel #4 at a flow rate of 100 ml/min.

9. With vessel #4 gently stirring, carefully pump in the contents from vessel #2 into vessel #4 at a flow rate of 100 ml/min. Try to pump the contents of vessel #2 near the stirring medium in vessel #4.

10. Based on the calculation in step 4, add the necessary weight of ethyl alcohol to a new vessel called #5.

11. Filter the contents of vessel #5 into vessel #6.

12. Pump the aqueous solution which is in vessel #4 and the organic solution which is in vessel #6 at flow rates of 20 ml/min and 5 ml/min, respectively, through a static mixer and into a 2 fluid nozzle which is placed on the spray drying chamber.

13. Powder is recovered approximately every hour from the baghouse filter bag.

The spray dried particles can be fabricated with a rough surface texture to modulate particle agglomeration and flowability of the powder. The spray-dried particle can be fabricated with features which enhance aerosolization via dry powder inhaler devices, and lead to lower deposition in the mouth, throat and inhaler device.

Methods and apparatus suitable for forming particles of the present invention are described in U.S. patent application entitled “Method and Apparatus for Producing Dry Particles”, filed concurrently herewith under Attorney Docket No. 00166.0115-US01, which is a Continuation-in-part of U.S. patent application Ser. No. 10/101,563 entitled “Method and Apparatus for Producing Dry Particles”, filed on Mar. 20, 2002, under the Attorney Docket No.00166.0115-US00. Methods and apparatus suitable for forming particles of the present invention are described in PCT patent application entitled “Method and Apparatus for Producing Dry Particles”, filed concurrently herewith under Attorney Docket No 00166.0115-WO01. The entire contents of these applications are incorporated by reference herein.

The solubility of an agent can have a significant effect on bioavailability regarding the rate and extent of absorption. Low solubility reduces the rate of dissolution of the agent, and thus reduces the rate and extent of uptake of the drug. Understanding of factors contributing to absorption efficiency of peptides and proteins delivered by inhalation remains far from complete. Extent of absorption is highly variable, ranging form 0 to 95% of dose administered. Although large proteins are generally absorbed at much slower rates than smaller peptides, molecular mass is clearly not the only factor involved since some poorly absorbed proteins are much smaller than well absorbed ones. Large proteins, in particular proteins in the ranges of 50-150 kD, are absorbed so poorly that designing suitable vehicles for pulmonary delivery is a unique challenge. Further, susceptibility of many proteins to surface denaturation, shear forces, oxidation, and aggregation makes their delivery via small particle aerosols even more challenging.

Additionally, removal of material deposited in the airways occurs not only by absorption, but also through mucociliary clearance. The rate and overall extent of loss of material from the lung via mucociliary clearance are affected by a number of variables, most importantly, the site of deposition (affected by particle size and shape). Thus, if aiming to optimize absorption, it is necessary to optimize deposition in the lower lung regions where ciliary clearance is slow or absent. For larger molecules, increased duration of residence in the lower or deep lung should lead to a greater opportunity for absorption from the alveoli.

Bioavailability is estimated by performing area under the curve (AUC) calculations. By increasing the percent composition by weight of hGH in the particles from about 50% to about 93% and by increasing the percentage of particles with an FPF <5.6 μm, Applicants have produced particles that are able to deliver more hGH to the lower lung regions thereby allowing for greater pulmonary absorption of hGH. Over the entire time course of the study (16 hours), the relative bioavailability of inhaled pulmonary hGH was approximately 6-8% relative to subcutaneously administered hGH.

The particles of the invention are employed in compositions suitable for drug delivery via the pulmonary system. For example, such compositions include the particles and a pharmaceutically acceptable carrier for administration to a patient, preferably for administration via inhalation. The particles can be co-delivered with larger carrier particles, not including a therapeutic agent, the latter possessing mass median diameters for example in the range between about 50 μm and about 100 μm. The particles can be administered alone or in any appropriate pharmaceutically acceptable carrier, such as a liquid, for example saline, or a powder, for administration to the respiratory system.

Particles including a medicament, for example one or more of the drugs listed above, are administered to the respiratory tract of a patient in need of treatment, prophylaxis or diagnosis. Administration of particles to the respiratory system can be by means such as known in the art. For example, particles are delivered from an inhalation device. In a preferred embodiment, particles are administered via a dry powder inhaler (DPI). Metered-dose-inhalers (MDI), nebulizers or instillation techniques also can be employed.

The methods of the invention also relate to administering to the respiratory tract of a subject, particles and/or compositions comprising the particles of the invention, which can be enclosed in a receptacle. As described herein, in certain embodiments, the invention is drawn to methods of delivering the particles of the invention, while in other embodiments, the invention is drawn to methods of delivering respirable compositions comprising the particles of the invention. As used herein, the term “receptacle” includes but is not limited to, for example, a capsule, blister, film covered container well, chamber and other suitable means of storing particles, a powder or a respirable composition in an inhalation device known to those skilled in the art. Receptacles containing the pharmaceutical composition are stored 2-8° C.

The receptacle can be used in a dry powder inhaler. Examples of dry powder inhalers that can be employed in the methods of the invention include but are not limited to, the inhalers disclosed in U.S. Pat. Nos. 4,995,385 and 4,069,819, the Spinhaler® (Fisons, Loughborough, U.K.), Rotahaler® (Glaxo-Wellcome, Research Triangle Technology Park, North Carolina), FlowCaps® (Hovione, Loures, Portugal), inhalators from Boehringer-Ingelheim, Germany, and the Aerolizer™ (Novartis, Switzerland), the Diskhaler (Glaxo-Wellcome, RTP, NC) and others known to those skilled in the art. In one embodiment, the inhaler employed is described in U.S. patent application Ser. No. 09/835,302, entitled “Inhalation Device and Method”, by David A. Edwards, et al., filed on Apr. 16, 2001 under Attorney Docket No. 00166.0109.US00 and in U.S. patent application Ser. No. 10/268,059, entitled “Inhalation Device and Method”, by David A. Edwards, et al., filed on Oct. 10, 2002. The entire contents of these applications are incorporated by reference herein.

The volume of the receptacle is at least about 0.37 cm³. For example, the volume of the receptacle is at least about 0.48 cm³, or at least about 0.67 cm³ or 0.95 cm³. The invention is also drawn to receptacles which are capsules, for example, capsules designated with a particular capsule size, such as 2, 1, 0, 00 or 000. Suitable capsules can be obtained, for example, from Shionogi (Rockville, Md.). Blisters can be obtained, for example, from Hueck Foils, (Wall, N.J.). Other receptacles and other volumes thereof suitable for use in the instant invention are known to those skilled in the art.

Stable pharmaceutical compositions are essential to maintain the effectiveness of the active agent. Stable compositions of spray dried particles containing hGH as the active agent were prepared. The stability can be measured by tests known to those skilled in the art over various time frames. Particularly relevant measurement of selected embodiments of the instant invention are: Refrigerated Stability ranging from at least 3 months to at least 2 years or more; and Room Temperature Stability ranging from at least 3 months to at least 1 year.

The key stability points include the (1) consistency in the rate of degradation is similar for all formulations, (2) minimization of the production of impurities during processing and (3) controlling the water content to affect the rate of degradation.

The receptacle encloses or stores particles and/or respirable compositions comprising particles. Such particles and/or respirable compositions comprising particles can be in the form of a powder. The receptacle is filled with particles and/or compositions comprising particles, as known in the art. For example, vacuum filling or tamping technologies may be used. Generally, filling the receptacle with powder can be carried out by methods known in the art. The particles, powder or respirable composition which is enclosed or stored in a receptacle has a mass of at least about 1 milligram. For example, the mass of the particles or respirable compositions stored or enclosed in the receptacle is at least about 5 milligrams, or at least about 10 milligrams, or at least about 15 milligrams, or at least about 20 milligrams, or at least about 25 milligrams. The receptacle and the inhalers are used in the recommended temperature of 5° C. to about 40° C. and at 15-95% relative humidity.

The receptacle encloses a mass of particles, especially a mass of highly dispersible particles as described herein. The mass of particles comprises a nominal dose of an agent. As used herein, the phrase “nominal dose” means the total mass of an agent which is present in the mass of particles in the receptacle and represents the maximum amount of agent available for administration in a single breath.

Particles and/or respirable compositions comprising particles are stored or enclosed in the receptacles and are administered to the respiratory tract of a subject. As used herein, the terms “administration” or “administering” of particles and/or respirable compositions refer to introducing particles to the respiratory tract of a subject. A plurality of receptacles can be provided in a kit, as further described in the Exemplification section below.

Methods of treating disease and delivering via the pulmonary system using these particles is also disclosed. In such methods, the particles possess rapid release properties. “Rapid release”, as that term is used herein, refers to an increased pharmacodynamic response typically seen in the first two hours following administration, and more preferably in the first hour. Rapid release also refers to a release of active agent, in particular inhaled hGH, in which the period of release of an effective level of agent is at least the same as, preferably shorter than that seen with presently available subcutaneous injections of active agent, in particular, Met-hGH and regular soluble hGH.

The rapid release particles are formulated using hGH and sodium phosphate monohydrate. The rapid release particles can further comprise a phospholipid. The rapid release is characterized by both the period of release being shorter and the levels of agent released being greater.

Alternatively, particles of the invention are capable of releasing bioactive agent in a sustained fashion. As such, the particles possess sustained release properties. “Sustained release”, as that term is used herein, refers to a reduction in the release of agent typically seen in first two hours following administration, and more preferably in the first hour, often referred to as the initial release. The sustained release is characterized by both the period of release being longer in addition to a decreased release. For example, a sustained release of hGH is a release showing elevated levels out to at least 4 hours post administration, such as about 6 hours or more.

Certain drugs pose special challenges due to the properties of the active agent coupled with the required amounts need for an effective dose. The particles of the instant invention are especially useful for administering hGH as the dosage required is high, ranging from 0.1 mg to 4.0 mg via subcutaneous injection. Compositions used in the methods of the invention comprising dry particles carrying surprisingly high loads of agent are also capable of targeting to particular regions of the respiratory system, for example, upper airways, central airways and/or deep lung. Formulations and methods of administering them are also described in U.S. application Ser. No. 09/591,307 (“High Efficient Delivery of a Large Therapeutic Mass Aerosol”) and Ser. No. 09/878,146 (“High Efficient Delivery of a Large Therapeutic Mass Aerosol”), filed, respectively on Jun. 9, 2000 and Jun. 8, 2001.

It is understood that the particles and/or respirable compositions comprising the particles of the invention which can be administered to the respiratory tract of a subject can also optionally include pharmaceutically-acceptable carriers, as are well known in the art. The term “pharmaceutically-acceptable carrier” as used herein, refers to a carrier which can be administered to a patient's respiratory system without any significant adverse toxicological effects. Appropriate pharmaceutically-acceptable carriers, include those typically used for inhalation therapy (e.g., lactose) and include pharmaceutically-acceptable carriers in the form of a liquid (e.g., saline) or a powder (e.g., a particulate powder). In one embodiment, the pharmaceutically-acceptable carrier comprises particles which have a mean diameter ranging from about 50 mm to about 200 μm, and in particular lactose particles in this range. It is understood that those of skill in the art can readily determine appropriate pharmaceutically-acceptable carriers for use in administering, accompanying and or co-delivering the particles of the invention.

Delivery to the pulmonary system of particles is in a single, breath-actuated step, as described, for example, in U.S. patent application, “High Efficient Delivery of a Large Therapeutic Mass Aerosol”, application Ser. No. 09/591,307, filed Jun. 9, 2000, which is incorporated herein by reference in its entirety. At least 85% of the mass of the particles stored in the inhaler receptacle, and at least 55% of the particles with an FPF less than 5.6 μm, is delivered to a subject's respiratory system in a single, breath-activated step. Alternatively, at least 1 milligram, or at least 10 milligrams, or even at least 25 milligrams of a medicament is delivered by administering, in a single breath, to a subject's respiratory tract particles enclosed in the receptacle. Amounts as high as 15, 20, 25, 30, 35, 40 and 50 milligrams can be delivered.

As used herein, the phrases “breath-activated” and “breath-actuated” are used interchangeably. As used herein, “a single, breath-activated step” means that particles are dispersed and inhaled in one step. For example, in single, breath-activated inhalation devices, the energy of the subject's inhalation both disperses particles and draws them into the oral or nasopharyngeal cavity. Suitable inhalers which are single, breath-actuated inhalers that can be employed in the methods of the invention are described above.

“Single breath” administration includes single, breath-activated administration, but also administration during which the particles, respirable compositions or powders are first dispersed, followed by the inhalation or inspiration of the dispersed particles, respirable compositions or powders. In the latter mode of administration, additional energy than the energy supplied by the subject's inhalation disperses the particles. An example of a single breath inhaler which employs energy other than the energy generated by the patient's inhalation is the device described in U.S. Pat. No. 5,997,848 issued to Patton et al. on Dec. 7, 1999, the entire teachings of which are incorporated herein by reference.

The receptacle enclosing the particles, respirable compositions comprising particles or powder is emptied in a single, breath-activated step, or in a single inhalation. As used herein, the term “emptied” means that at least 50% of the particle mass enclosed in the receptacle is emitted from the inhaler during administration of the particles to a subject's respiratory system. For example, at least 85% of the particle mass enclosed in the receptacle and at least 90% of the particles with an FPF less than 5.6 μm, is emitted from the inhaler during administration of the particles to a subject's respiratory system.

Particles administered to the respiratory tract travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. The particles of the invention are designed such that upon administration the particles are delivered to specific regions of the lung. For example, most of the mass of particles deposit in the deep lung, or delivery of the particles is primarily to the central airways, or to the upper airways.

Delivery to the pulmonary system of particles in a single, breath-actuated step is enhanced by employing particles which are dispersed at relatively low energies, such as, for example, at energies typically supplied by a subject's inhalation. Such energies are referred to herein as “low”. As used herein, “low energy administration” refers to administration wherein the energy applied to disperse and inhale the particles is in the range typically supplied by a subject during inhaling.

As used herein, the term “effective amount” means the amount needed to achieve the desired therapeutic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol).

The term “dose” of growth hormone refers to that amount that provides therapeutic effect in an administration regimen. A dose may consist of more than one actuation. The formulations hereof are prepared containing amounts of hGH, for example, but not limited to, from about 0.1 mg to about 40 mg, or from about 0.1 mg to about 25 mg, or from 0.1 mg to about 5 mg, calculated on the ready-to-use formulation. For use of these compositions in administration to human beings suffering from hypopituitary dwarfism, for example, these formulations contain from about 0.1 mg to about 10 mg, corresponding to the currently contemplated dosage regimen for the intended treatment. The concentration range is not critical to the invention and may be varied by the physician supervising the administration.

Aerosol dosage, formulations and delivery systems also may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

The particles of the invention have specific drug release properties. Release rates can be controlled as described below and as further described in U.S. application Ser. No. 09/644,736 filed Aug. 23, 2000 entitled “Modulation of Release From Dry Powder Formulations” by Sujit Basu, et al., which is incorporated herein by reference.

Drug release rates can be described in terms of the half-time of release of a bioactive agent from a formulation. As used herein the term “half-time” refers to the time required to release 50% of the initial drug payload contained in the particles. Fast or rapid drug release rates generally are less than 30 minutes and range from about 1 minute to about 60 minutes.

Drug release rates can be described in terms of release constants. The first order release constant can be expressed using the following equations:

M₍ₜ₎=M_{(∞)}* (1−e^{-k*t}) (1)

Where k is the first order release constant. M_{(∞)} is the total mass of drug in the drug delivery system, e.g. the dry powder, and M₍ₜ₎ is the amount of drug mass released from dry powders at time t.

Equations (1) may be expressed either in amount (i.e., mass) of drug released or concentration of drug released in a specified volume of release medium. For example, Equation (1) maybe expressed as:

C₍ₜ₎=C_{(∞)}* (1−e^{-k*t}) or Release₍ₜ₎=Release_{(∞)* (}1−e^{-k*t}) (2)

Where k is the first order release constant. C_{(∞)} is the maximum theoretical concentration of drug in the release medium, and C₍ₜ₎ is the concentration of drug being released from dry powders to the release medium at time t.

Drug release rates in terms of first order release constant can be calculated using the following equations:

k=−ln(M_{(∞)}−M₍ₜ₎)/M_{(∞)}/t (3)

As used herein, the term “a” or “an” refers to one or more.

The term “nominal dose” as used herein, refers to the total mass of bioactive agent which is present in the mass of particles targeted for administration and represents the maximum amount of bioactive agent available for administration.

While this invention has been particularly shown and described with references to preferred embodiments thereof, it will be understood by those skilled in the art that various changes in form and details may be made therein without departing from the scope of the invention encompassed by the appended claims.

Exemplification

Equipment and materials used in the preparation and characterization of particles is listed below.

(1) RODOS dry powder disperser (Sympatec Inc., Princeton, N.J.)

(2) HELOS laser diffractometer (Sympatec Inc., N.J.)

(3) AeroDisperser (TSI, Inc., Amherst, Mass.)

(4) Aerosizer (TSI Inc., Amherst, Mass.)

(5) blister pack machine, Fantasy Blister Machine (Schaefer Tech, Inc., Indianapolis, Ind.)

(6) collapsed Andersen Cascade Impactor (consisting of stage 0, 2 and F as defined by manufacturer) and the filter stage (Thermo Anderson Inst., Smyrna, Ga.)

(7) a multi-stage liquid Impinger (MSLI) (Erweka, USA, Milford, Conn.)

(8) suitable static mixers and apparatus for spray drying are described above.

Reagents

human growth hormone (Eli Lilly, Indianapolis, Ind.)

Sodium Phosphate Monohydrate (Spectrum Chemicals, NJ)

Ammonium Bicarbonate (Spectrum Chemicals, NJ)

Ethanol

hydroxypropyl methyl cellulose capsules (Shionogi, Japan)

blister packs (Heuck Foils, Wall, N.J)

DPPC (Genzyme, Cambridge, Mass.)

Mass Median Aerodynamic Diameter-MMAD (μm)

The mass median aerodynamic diameter was determined using an Aerosizer/Aerodisperser (Amherst Process Instrument, Amherst, Mass.). Approximately 2 mg of powder formulation was introduced into the Aerodisperser and the aerodynamic size was determined by time of flight measurements.

Volume Median Geometric Diameter-VMGD (μm)

The volume median geometric diameter was measured using a RODOS dry powder disperser (Sympatec, Princeton, N.J.) in conjunction with a HELOS laser diffractometer (Sympatec). Powder was introduced into the RODOS inlet and aerosolized by shear forces generated by a compressed air stream regulated at 2 bar. The aerosol cloud was subsequently drawn into the measuring zone of the HELOS, where it scattered light from a laser beam and produced a Fraunhofer diffraction pattern used to infer the particle size distribution and determine the median value.

Aerosol Performance:

Gravimetric analysis, using Cascade impactors, is a method of measuring the size distribution of airborne particles. The Anderson Cascade Impactor (ACI) is an eight-stage impactor that can separate aerosols into nine distinct fractions based on aerodynamic size. For this project a two-stage collapsed ACI, a three-stage ACI (wetted or dry) and/or an eight-stage ACI (wetted or dry) can be used.

### EXAMPLE 1

A. 93.5 wt % hGH/6.5 wt % Sodium Phosphate

Lipid-free particles with a formulation containing hGH and sodium phosphate were prepared as follows. The aqueous solution was prepared by preparing a bulk sodium phosphate solution at 100 mM at pH 7.4 and a bulk ammonium bicarbonate solution at 50 g/L. 52 ml of 100 mM sodium phosphate buffer at pH 7.4 was added to 268 ml of water for irrigation. To this was added 200 ml of the 50 g/L ammonium bicarbonate solution and 200 ml of ethanol. The resulting solution was combined in a static mixer with 280 mL of bulk hGH at 40 g/L in 1.7 mM sodium phosphate buffer at pH=7.4. Solute concentration in the combined solution was 12 g/L. The combined solution was spray dried under the following process conditions:

Inlet temperature ˜ 74° C.
Outlet temperature from the drying drum ˜ 40° C.
Nitrogen drying gas = 110 kg/hr
Nitrogen atomization gas = 64 g/min
2 Fluid internal mixing nozzle atomizer
Nitrogen atomization pressure ˜ 90 psi
Liquid feed rate = 25 ml/mm
Liquid feed temperature ˜ 22° C.
Pressure in drying chamber = −2.0 in water

The resulting particles had a FPF(5.6) of 75%, and a FPF(3.4) of 70%, both measured using a 2-stage ACT. The volumetric median geometric diameter (VMGD) was 8 μm at 1.0 bar. The resulting particles had a soluble dimer fraction of 1.2% and a readily extractable hGH fraction of 97.5%.

The combination solution flowing out of the static mixer was fed into a two-fluid nozzle atomizer. The contact between the atomized droplets from the atomizer and the heated nitrogen caused the liquid to evaporate from the droplets, resulting in dry porous particles. The resulting gas-solid stream was fed to product filter that retained the resulting dry particles, and allowed the hot gas stream containing the drying gas (nitrogen), evaporated water, and ethanol to pass. The dry particles were collected into a product collection vessel.

In order to obtain dry particles of particular physical and chemical characteristics, in vitro characterization tests can be carried out on the finished dry particles, and the process parameters adjusted accordingly, as described above. Particles containing 93.5 wt % hGH and 6.5 wt % sodium phosphate produced using this method had a VMGD of 8.0 μm, FPF(5.6) of 75%, readily extractable hGH fraction of 97.5%, and a soluble dimer fraction of 1.2%.

B. 93.5 wt % hGH/6.5 wt % Sodium Phosphate

Lipid-free particles with a formulation containing hGH and sodium phosphate were prepared as follows. The aqueous solution was prepared by dissolving 0.78 g sodium phosphate dibasic in 500 ml of Water for Irrigation (WFI). To this was added 11.74 g bulk hGH lyophilization powder with a water content of 4.4%. The organic solution was prepared by dissolving 30 g of ammonium bicarbonate in 300 ml of water for irrigation, then combining the ammonium bicarbonate solution with 200 ml of ethanol. The aqueous solution, at a pH of approximately 7.0 and the organic solution were combined in a static mixer prior to being introduced to the spray dryer nozzle. Solute concentration in the combined solution was 12 g/L. The combined solution was spray dried under the following process conditions:

Inlet temperature ˜ 74° C.
Outlet temperature from the drying drum ˜ 40° C.
Nitrogen drying gas = 110 kg/hr
Nitrogen atomization gas = 80 g/min
2 Fluid internal mixing nozzle atomizer
Nitrogen atomization back pressure ˜ 100 psi
Liquid feed rate = 25 ml/min
Liquid feed temperature ˜ 22° C.
Pressure in drying chamber = −2.0 in water

The resulting particles had a FPF(3.3) of 69%, measured using a three-stage, wetted screen, ACI. The VMGD was 7.0 μm at 1.0 bar. The resulting particles had a soluble dimer fraction of 1.5% and a readily extractable hGH fraction of 96%.

The combination solution flowing out of the static mixer was fed into a two-fluid nozzle atomizer. The contact between the atomized droplets from the atomizer and the heated nitrogen caused the liquid to evaporate from the droplets, resulting in dry porous particles. The resulting gas-solid stream was fed to product filter that retained the resulting dry particles, and allowed the hot gas stream containing the drying gas (nitrogen), evaporated water, and ethanol to pass. The dry particles were collected into a product collection vessel.

In order to obtain dry particles of particular physical and chemical characteristics, in vitro characterization tests can be carried out on the finished dry particles, and the process parameters adjusted accordingly, as described above. Particles containing 93.5 wt % hGH and 6.5 wt % sodium phosphate produced using this method had a VMGD of 7.0 μm, FPF(3.3) of 69%, readily extractable hGH fraction of 96%, and a soluble dimer fraction of 1.5%.

### EXAMPLE 2

93 wt % hGH/7 wt % Sodium Phosphate

Particles with a formulation comprising hGH and sodium phosphate were prepared as follows. A 14 g/L bulk hGH/sodium phosphate solution was prepared by dissolving hGH in 1.7 mM sodium phosphate buffer at pH 7.4. The pH was maintained at 7.4 by adding 1.0 N NaOH. The aqueous solution was prepared by adding 328 mg sodium phosphate monobasic to 400 ml of water for irrigation, adjusting the pH to 7.4 using 1.0 N NaOH. To this was added 15 g ammonium bicarbonate solution and 400 ml of the 14 g/L hGH bulk solution. The organic solution comprised 200 ml ethanol. The aqueous solution and the organic solution were combined in a static mixer. Solute concentration in the combined solution was 6 g/L. The combined solution was spray dried under the following process conditions:

Inlet temperature ˜ 115° C.
Outlet temperature from the drying drum ˜ 70° C.
Nitrogen drying gas = 110 kg/hr
Nitrogen atomization gas = 46 g/min
2 Fluid internal mixing nozzle atomizer
Nitrogen atomization pressure ˜ 65 psi
Liquid feed rate = 25 ml/min
Liquid feed temperature ˜ 22° C.
Pressure in drying chamber = −2.0 in water

The resulting particles had a FPF(5.6) of 84%, and a FPF(3.4) of 77%, both measured using a 2-stage ACI. The volume mean geometric diameter was 9.6 μm at 1.0 bar. The resulting particles had a soluble dimer fraction of 4.0% and a readily extractable hGH fraction of 97.7%.

The combination solution flowing out of the static mixer was fed into a two-fluid nozzle atomizer. The contact between the atomized droplets from the atomizer and the heated nitrogen caused the liquid to evaporate from the droplets, resulting in dry porous particles. The resulting gas-solid stream was fed to bag filter that retained the resulting dry particles, and allowed the hot gas stream containing the drying gas (nitrogen), evaporated water, and ethanol to pass. The dry particles were collected into a product collection vessel.

In order to obtain dry particles of particular physical and chemical characteristics, in vitro characterization tests can be carried out on the finished dry particles, and the process parameters adjusted accordingly, as described above. Particles containing 93 wt % hGH, and 7 wt % sodium phosphate produced using this method had a VMGD of 9.6 μm, FPF(5.6) of 84%, readily extractable hGH fraction of 97.7%, and a soluble dimer fraction of 4.0%.

Through the process of the present invention, the formation of protein aggregates can be minimized. Reduced protein aggregation is achieved through the use of the static mixer, and by controlling the level of ethanol in the ethanol solution.

### EXAMPLE 3

80 wt %hGH/14 wt %DPPC/6 wt % Sodium Phosphate

Particles with a formulation comprising hGH, DPPC, and sodium phosphate were prepared as follows. A 14 g/L bulk hGH/sodium phosphate solution was prepared by dissolving hGH in 1.7 mM sodium phosphate buffer at pH 7.4. The pH was maintained at 7.4 by adding 1.0 N NaOH. The aqueous solution was prepared by adding 280 mg sodium phosphate monobasic to 457 ml of water for irrigation. To this was added 15 g ammonium bicarbonate solution and 343 ml of the 14 g/L hGH bulk solution. The organic solution was prepared by adding 840 mg DPPC to 200 ml ethanol. The aqueous solution and the organic solution were combined in a static mixer. Solute concentration in the combined solution was 6 g/L. The combined solution was spray dried under the following process conditions:

Inlet temperature ˜ 120° C.
Outlet temperature from the drying drum ˜ 70° C.
Nitrogen drying gas = 110 kg/hr
Nitrogen atomization gas = 40 g/min
2 Fluid internal mixing nozzle atomizer
Nitrogen atomization pressure ˜ 65 psi
Liquid feed rate = 30 ml/min
Liquid feed temperature ˜ 22° C.
Pressure in drying chamber = −2.0 in water

The resulting particles had a FPF(5.6) of 89%, and a FPF(3.4) of 76%, both measured using a 2-stage ACI. The volume mean geometric diameter was 7.4 μm at 1.0 bar. The resulting particles had a soluble dimer fraction of 3.5% and a readily extractable hGH fraction of 95.6%.

The combination solution flowing out of the static mixer was fed into a two-fluid nozzle atomizer. The contact between the atomized droplets from the atomizer and the heated nitrogen caused the liquid to evaporate from the droplets, resulting in dry porous particles. The resulting gas-solid stream was fed to bag filter that retained the resulting dry particles, and allowed the hot gas stream containing the drying gas (nitrogen), evaporated water, and ethanol to pass. The dry particles were collected into a product collection vessel.

In order to obtain dry particles of particular physical and chemical characteristics, in vitro characterization tests can be carried out on the finished dry particles, and the process parameters adjusted accordingly, as described above. Particles containing 80 wt % hGH, 14 wt % DPPC and 6 wt % sodium phosphate produced using this method had a VMGD of 7.4 μm, FPF(5.6) of 89%, readily extractable hGH fraction of 95.6%, and a soluble dimer fraction of 3.5%.

Through the process of the present invention, the formation of protein aggregates can be minimized. Reduced protein aggregation is achieved through the use of the static mixer, and by controlling the level of ethanol in the ethanol solution.

### EXAMPLE 4

Study for Growth Hormone Inhalation Powder Kit

12 individuals were chosen for the clinical trials of the hGH Inhalation Powder Kit. Each individual was given an inhaler, for example, an inhaler as described in U.S. patent application Ser. No. 09/835,302, entitled “Inhalation Device and Method”, by David A. Edwards, et al., filed on Apr. 16, 2001 under Attorney Docket No. 00166.0109.US00. Each individual was instructed to inhale a hGH formulation as follows.

Preparation

The mouthpiece was removed from the inhaler body to allow access to the capsule chamber. The number of growth hormone capsules that are required for the dose were removed from the blister package. The hGH capsules were at room temperature for at least one hour but not more than three hours. One growth hormone capsule was inserted into the capsule chamber. The mouthpiece was reattached onto inhaler body by pressing both pieces firmly together until a snap was heard and the motion stopped. This action punctured the capsule, making it ready to use.

Administration

Before beginning, the subject needed to ensure that the mouth was clear of any potential obstructions. The individuals were instructed to sit upright, relax and breathe normally for at least five breaths, then remove the inhaler cap. The individuals were then instructed to hold the inhaler away from their mouths, and exhale as much as possible without becoming uncomfortable, and without forcing their breath out. Then they inserted the mouthpiece into their mouths, making sure the inhaler was held straight out from the mouth and horizontal. They then took a deep breath “in” through their mouths—until their lungs were full—removing mouthpiece and holding their breath for five seconds, then letting it out normally.

Capsule Inspection and Disposal

The mouthpiece was removed from the inhaler body, and the capsule was removed from the chamber. The capsule was inspected to make sure the dose was administered. Generally, the capsule had a light dusting of white powder on the inside and two (2) holes on the bottom. If more than a light dusting of powder remained in the capsule, the capsule was reinserted back into the capsule chamber and administration was repeated until all the powder (except the normal dusting) was inhaled. (When reinserting the capsule, the end of the capsule with two (2) holes was placed into the chamber first.)

Storing the Kit

The remaining contents were returned to its case. The case with the remaining capsules was stored in the refrigerator at the recommended storage conditions (2° C./36° F. −8° C./46° F.).

Safety Results

Subjects were assessed for cough, gagging and abnormal taste after pulmonary dosing. Vital signs and pulmonary function measured up to 12 hours after dosing. Subjects were monitored for clinically significant changes. Adverse Events (AEs) were recorded.

### EXAMPLE 5

Particles of the instant invention were administered as in Example 4. The data was then collected for each of the 12 subjects who were 12 healthy males. Pulmonary formulations comprising, by weight, 93% hGH and 7% sodium phosphate (F3) and 80% hGH, 14% DPPC and 6% sodium phosphate (F2) were well tolerated in the 12 subjects. Relative bioavailability compared to subcutaneous administration was approximately 6-7% (F2) and 7-8% (F3) respectively. Inhaled doses of F2 (74 mg) and F3 (78.4 mg) produce similar peak hGH concentrations and systemic exposure to subcutaneous 4 mg. Mean inspiratory flow rate was 0.84 L/sec (range 0.64 to 1.06 L/sec).

As mentioned, the subjects were assessed for cough, gagging and abnormal taste after pulmonary dosing. Their vital signs and pulmonary function measured up to 12 hours after dosing. There were no clinically significant changes. Data on Adverse Events (AEs) was collected. AEs were reported by ten (10) subjects, principally headache five (5), nausea one (1), and postural dizziness two (2). No coughing or issues with taste reported.

### EXAMPLE 6

Nozzles

a) Two-Fluid Nozzle—Single-Hole.

The two-fluid, single-hole nozzle can be either an internal mixing nozzle or an external mixing nozzle. The two-fluid, single-hole nozzle that is currently used is an internal mixing nozzle. Two-fluid atomization involves impacting liquid bulk with high-velocity gas. The high-velocity gas creates high frictional forces over liquid surfaces causing liquid disintegration into spray droplets. The liquid feed is pumped through an orifice into a sloped chamber where it is contacted by and mixed with the atomization gas. The combined atomization gas and the feed are forced through an orifice into the spray dryer.

TABLE 1
Solution and Process Conditions for Single-Hole Nozzle.

| | | |
|---|---|---|
| Feed solution | Solids Concentration | 12 g/L |
| | Ammonium Bicarbonate conc. | 30 g/L |
| | Solvent: Ethanol/Water (vol/vol %) | 20/80 |
| Process conditions | Feed Rate | 25 mL/min |
| | Atomization Gas Rate | 80 g/min |
| | Drying Gas Rate | 110 kg/hr |
| | Spray Dryer Outlet Temperature | 40° C. |

TABLE 2
Reproducibility Runs for Single-Hole Nozzle.

| | | | | | VMGD by | | | | |
|---|---|---|---|---|---|---|---|---|---|
| | Total | | | | RODOS (μm) | | | FPF < 3.3 μm | |
| | hGH | HMWP | RE | Water | 0.5 | 1.0 | 2.0 | by ACI-3 (%) | |
| | (%) | (%) | (%) | (%) | bar | bar | bar | 28.3 1 pm | 60 1 pm |
| | 87.3 | 1.2 | 95.4 | 6.7 | 6.1 | 5.3 | 4.9 | — | 75 |
| | 83.8 | 1.1 | 96.7 | — | 8.5 | 7.4 | 6.6 | — | 70 |
| | 82.6 | 1.2 | 95.5 | — | 6.7 | 5.6 | 5.0 | — | 70 |
| | 85.1 | 1.6 | 95.4 | 6.9 | 6.7 | 6.1 | 5.0 | 67 | 70 |
| | 87.1 | 1.7 | 94.4 | 6.4 | 6.5 | 5.3 | 4.4 | — | 75 |
| | 87.9 | 1.3 | 95.6 | — | 7.5 | 6.9 | 6.2 | — | 61 |
| | 86.9 | 1.4 | 95.9 | 6.2 | 7.8 | 6.9 | 6.0 | — | 63 |
| | 83.6 | 1.1 | 97.5 | 5.9 | 8.0 | 6.8 | 6.0 | — | 70 |
| | 83.6 | 2.4 | 96.6 | — | 9.4 | 8.1 | 6.4 | — | 72 |
| | 81.2 | 1.5 | 96.6 | 6.3 | 7.1 | 6.4 | 5.9 | — | 69 |
| | 81.7 | 1.5 | 96.8 | 6.2 | 7.3 | 6.2 | 5.8 | — | 68 |
| | 84.1 | 1.8 | 95.2 | 6.0 | 8.4 | 7.1 | 6.5 | — | 63 |
| | 84.7 | 1.4 | 96.1 | 6.3 | 8.8 | 7.7 | 6.8 | 67 | — |
| | 84.1 | 1.5 | 95.2 | 5.8 | 8.8 | 7.5 | 6.5 | 73 | — |
| Average | 84.6 | 1.5 | 95.9 | 6.3 | 7.7 | 6.7 | 5.9 | 69 | 69 |
| StDev | 2.0 | 0.3 | 0.8 | 0.3 | 1.0 | 0.9 | 0.7 | 3 | 4 |
| Range | 81.2- | 1.1- | 94.4- | 5.8- | 6.1- | 5.3- | 4.4- | 67- | 61- |
| | 87.9 | 2.4 | 97.5 | 6.9 | 9.4 | 8.1 | 6.8 | 73 | 75 |

B. Two-Fluid Nozzle—Six-Hole

The two-fluid six-hole nozzle operates under the same principles as the single-hole nozzle, except that the air cap has 6 holes. The six-hole nozzle generally produced powders with a larger geometric size and lower density than those produced with the single-hole nozzle. The six-hole nozzle can also process higher solids concentrations which increases production rates and helps with readily extractable values.

TABLE 3
Solution and Process Conditions for Six-Hole Nozzle.

| | | |
|---|---|---|
| Feed solution | Ammonium Bicarbonate cone. | 30 g/L |
| | Solvent: Ethanol/Water (vol/vol %) | 20/80 |
| Process conditions | Atomization Gas Rate | 120 g/min |
| | Drying Gas Rate | 110 kg/hr |
| | Spray Dryer Outlet Temperature | 45° C. |

TABLE 4
Physical and Chemical Characteristics for Six-Hole Nozzle.

| | | Liquid | | | | | |
|---|---|---|---|---|---|---|---|
| | Solids | feed rate | HMWP | RE | VMGD | FPF_{TD} < | |
| Nozzle | conc (g/L) | (mL/min) | (%) | (%) | 1 bar (μm) | 3.3 μm | Method |
| Six-hole | 30 | 10 | 1.9 | 97.7 | 8.2 | 66 | ACI-3, 60 1 pm |
| Six-hole | 30 | 20 | 1.7 | 97.7 | 9.3 | 63 | ACI-3, 60 1 pm |
| Six-hole | 60 | 10 | 1.5 | 97.4 | 7.3 | 57 | ACI-3, 60 1 pm |
| Six-hole | 60 | 20 | 1.6 | 97.9 | 8.8 | 58 | ACI-3, 60 1 pm |

Two design of experiments (DOE) were completed using the six-hole nozzle to explore a range of process conditions. The goal of these experiments was to narrow in on the optimal process conditions. Table 5 shows the ranges used for each of the variables in the two DOEs. The ranges for the second design were based on the data analysis of the first set of experiments. JMP v.4 software from SAS Institute, Cary, N.C. was used to analyze the data. The results of both DOEs are shown in Table 6. The main results from these experiments were that a higher atomization gas rate, and thus a higher atomization gas to liquid feed rate ratio, contributed to higher FPF values. Higher solids concentrations also contribute to higher readily extractable values. Higher outlet temperatures created dimer concentrations greater than the target of 2%.

TABLE 5
Variables in Six-Hole Nozzle DOEs.

| Variable | DOE-1 | DOE-2 |
|---|---|---|
| Ammonium Bicarbonate Conc., g/L | 30-40 | 5-30 |
| Solids Conc., g/L | 15-25 | 15-30 |
| Atomization Gas, g/min | 50-90 | 70-100 |
| Liquid Feed Rate, mL/min | 25-40 | 20-40 |
| Spray Dryer Outlet Temp., ° C. | 40-60 | 45-65 |

TABLE 6
Six-Hole Nozzle DOE Results.

| | | | | | | | | | | | | FPF < 3.3 |
|---|---|---|---|---|---|---|---|---|---|---|---|---|
| Amm | | | | SD | | | | | VMGD by | | | μm by |
| Bicarb | Solids | Atom. | Liquid | Outlet | Total | HM | | | RODOS (μm) | | | ACI-3 |
| Conc | Conc. | Gas | Feed Rate | Temp | hGH | WP | RE | Water | 0.5 | 1.0 | 2.0 | (%) |
| (g/L) | (g/L) | (g/min) | (mL/min) | (° C.) | (%) | (%) | (%) | (%) | bar | bar | bar | 60 1 pm |
| First | | | | | | | | | | | | |
| DOE | | | | | | | | | | | | |
| 30 | 15 | 50 | 25 | 60 | 85.0 | 1.9 | 96.1 | 5.6 | 14.2 | 12.4 | 11.2 | 49 |
| 30 | 15 | 90 | 40 | 60 | 84.6 | 1.8 | 94.9 | 4.9 | 14.6 | 11.8 | 10.3 | 65 |
| 40 | 15 | 90 | 40 | 40 | 82.1 | 1.3 | 94.7 | 8.0 | 23.5 | 19.6 | 14.9 | 56 |
| 40 | 15 | 50 | 40 | 40 | 82.7 | 1.0 | 94.8 | 8.4 | 17.9 | 15.1 | 12.8 | 44 |
| 40 | 25 | 90 | 25 | 60 | 83.2 | 3.4 | 97.4 | 5.0 | 11.4 | 10.2 | 9.5 | 66 |
| 30 | 25 | 50 | 40 | 40 | 81.3 | 2.5 | 97.2 | 8.0 | 14.9 | 13.1 | 11.1 | 53 |
| 40 | 25 | 90 | 40 | 60 | 84.3 | 3.0 | 97.3 | 5.3 | 16.0 | 15.1 | 11.9 | 43 |
| 30 | 25 | 50 | 25 | 40 | 82.8 | 2.4 | 97.6 | 6.3 | 14.2 | 12.9 | 11.1 | 44 |
| 35 | 25 | 70 | 32.5 | 50 | 83.7 | 1.7 | 97.0 | 5.3 | 14.8 | 13.3 | 11.8 | 53 |
| Second | | | | | | | | | | | | |
| DOE | | | | | | | | | | | | |
| 30 | 30 | 100 | 40 | 65 | 82.2 | 1.7 | 97.3 | 5.8 | 19.3 | 15.3 | 12.4 | 46 |
| 30 | 30 | 70 | 20 | 65 | 86.6 | 1.7 | 97.4 | 5.0 | 17.5 | 15.4 | 10.9 | 49 |
| 5 | 15 | 70 | 20 | 65 | 85.1 | 1.4 | 97.3 | 4.6 | 20.0 | 16.0 | 11.8 | 49 |
| 30 | 15 | 70 | 40 | 45 | 82.7 | 1.1 | 97.4 | 6.0 | 18.5 | 16.5 | 14.8 | 48 |
| 5 | 30 | 100 | 20 | 45 | 86.4 | 0.9 | 97.9 | 5.8 | 17.1 | 12.6 | 10.8 | 56 |
| 5 | 15 | 100 | 40 | 65 | 85.3 | 1.0 | 96.4 | 5.1 | 30.3 | 23.3 | 18.5 | 30 |
| 5 | 30 | 70 | 40 | 45 | 86.1 | 0.4 | 98.1 | 6.2 | 26.7 | 22.4 | 18.9 | 34 |
| 17.5 | 22.5 | 85 | 30 | 55 | 86.9 | 0.8 | 97.0 | 5.5 | 23.8 | 18.7 | 15.1 | 46 |
| 30 | 15 | 100 | 20 | 45 | 83.7 | 1.2 | 97.0 | 5.7 | 15.5 | 12.7 | 11.6 | 65 |

### EXAMPLE 7

Addition of Tween to the Formulation Solution.

The addition of non-ionic surfactants at optimum concentrations to hGH containing solutions has been demonstrated in the literature to significantly reduce the formation of insoluble aggregates during exposure to an air-liquid interface (Bam et al., 1998; Pearlman and Bewley, 1993; Katakam et al., 1995). Non-ionic surfactants, such as Tween, preferentially adsorb to air-water interfaces and stabilize proteins against aggregation during processing (filtration, spray-drying, mixing, reconstitution).

The amount of Tween-20 and -80 added to the spray-drying solution, containing 20% ethanol and 8 g/L solids, was studied between 4.0% and 0.5% weight per weight of solids. The solutions were agitated to induce aggregation. The amount of aggregation was assessed before and after agitation. Results are shown in Table 7. The optimum percent of Tween added to the formulation to reduce soluble aggregates is between 1.0 and 0.5%, which translates to a solution concentration of 0.008% and 0.004% which is lower than reported in the literature. At the Tween concentration of 0.008%, the solutions were clear which indicates the presence of no insoluble aggregates. A slightly turbid solution was observed at 0.004% which indicates very low levels of insoluble aggregates. At concentrations of 0.0008% and 0.0002% the solutions were significantly more turbid indicating increased insoluble aggregation.

TABLE 7
Effect of Tween in the Spray-Drying Formulation Solution.

| | | HMW Protein in Solution | | | |
|---|---|---|---|---|---|
| Tween | | Tween-20 | | Tween-80 | |
| Solution | Tween | Before | After | Before | After |
| Concentration | w/w of solids | Agitation | Agitation | Agitation | Agitation |
| 0.0320% | 4.00% | 0.73% | 3.86% | 1.32% | 4.84% |
| 0.0160% | 2.00% | 0.66% | 3.76% | 0.88% | 4.47% |
| 0.0080% | 1.00% | 0.61% | 3.54% | 0.76% | 4.31% |
| 0.0040% | 0.50% | 0.59% | 3.70% | 0.66% | 4.33% |
| 0.0008% | 0.10% | 0.55% | 4.34% | 0.65% | 5.53% |
| 0.0002% | 0.02% | 0.62% | 5.27% | 0.63% | 6.76% |

A further solution was spray-dried containing 12 g/L solids (93% hGH, 7% sodium phosphate) in 20% ethanol having 20 g/L of volatile ammonium bicarbonate. This solution contained either 0, 2.8, 5.6 or 11.2% Tween-80 (w/w of solids). Table 8 shows a significant increase in Readily Extractable hGH upon the addition of 2.8, 5.6 or 11.2% Tween-80 to the formulation.

TABLE 8
Effect of Tween on Spray-Dried Powder.

| Readily | | |
|---|---|---|
| Extractable | Tween-80 | Tween-80 |
| hGH | Concentration | (w/w solids) |
| 97.9% | 0.000% | 0.00% |
| 99.7% | 0.033% | 2.8% |
| 99.8% | 0.066% | 5.6% |
| 99.9% | 0.132% | 11.2% |

### EXAMPLE 8

Solids Concentration.

Solids concentration is the total concentration of hGH plus non-volatile excipients used in the formulation solution. The solids concentration has a definitive effect on the level of protein aggregation in the spray-dried powder. As the solids or hGH concentration is increased, a significant decrease in insoluble aggregates, an increase in readily extractable hGH and a reduction in FPF is obtained. The increase in readily extractable protein is believed to be due to a percent reduction in exposure of the hGH at the air/liquid interface that is known to cause insoluble aggregates. Because there is a fixed amount of aggregation at the air/liquid interface for a particular formulation or spray-drying conditions, an increase in total solids causes a percent decrease in hGH insoluble aggregates. A benefit of higher solids concentration is a more efficient powder production. As used herein, the range of solids concentration for the single-hole nozzle was 6-30 g/L and for the six-hole nozzle was 6-60 g/L. Representative results for this example are set forth in Table 9.

TABLE 9

| | Solids | HMWP | RE | VMGD | FPF | |
|---|---|---|---|---|---|---|
| Nozzle | conc (g/l) | (%) | (%) | 1 bar (μm) | (%) | Method |
| Single-hole | 8 | 3.2 | 98.2 | 6.1 | 82 | ACI-2, 60 1 pm |
| Single-hole | 12 | 1.8 | 98.2 | 7.3 | 69 | ACI-2, 60 1 pm |
| Single-hole | 12 | 1.5 | 97.7 | 8.2 | 77 | ACI-2, 60 1 pm |
| Single-hole | 30 | 1.1 | 96.1 | 6.2 | 65 | ACI-2, 60 1 pm |
| Six-hole | 15 | 1.2 | 97.0 | 12.7 | 65 | ACI-3, 60 1 pm |
| Six-hole | 60 | 1.6 | 97.9 | 8.8 | 58 | ACI-3, 60 1 pm |

### EXAMPLE 9

Ammonium Bicarbonate Concentration.

Ammonium bicarbonate is used as a volatile solid in the spray drying solution to help achieve desirable physical characteristics in the final particles. As the concentration of ammonium bicarbonate increases, FPF and powder dispersibility improve. However, volatile salts prove a challenge to the chemical integrity of the hGH since as the salts effervesce, they further increase the liquid surface area during the process. Removing the ammonium bicarbonate from the spray-drying solution eliminates this rapid volatilization of gas during drying and reduces the subsequent formation of hGH aggregates. Additionally, the higher levels seem to increase the HMWP and decrease the readily extractable protein. As used herein, the range of ammonium bicarbonate concentration for the single-hole nozzle was 0-30 g/L and for the six-hole nozzle was 0-40 g/L. Representative results for this example are set forth in Table 10.

TABLE 10

| | Amm | | | | | |
|---|---|---|---|---|---|---|
| | Bicarb | HMWP | RE | VMGD | FPF | |
| Nozzle | Conc (g/l) | (%) | (%) | 1 bar (μm) | (%) | Method |
| Single- | 10 | 1.1 | 97.9 | 9.1 | 69 | ACI-2, 60 1 |
| hole | | | | | | pm |
| Single- | 29 | 2.0 | 96.6 | 7.6 | 77 | ACI-2, 60 1 |
| hole | | | | | | pm |
| Single- | 0 | 1.2 | 95.5 | 12.4 | 52 | ACI-3, 60 1 |
| hole | | | | | | pm |
| Single- | 30 | 1.2 | 95.5 | 5.6 | 70 | ACI-3 60 1 |
| hole | | | | | | pm |

### EXAMPLE 10

Solvent Ratios.

The inclusion of alcohol as a co-solvent to the aqueous phase serves to help achieve desired physical characteristics. An optimal level of alcohol can also reduce protein aggregation at the air-liquid interface during the AIR spray drying process, however a level too high can cause detrimental protein structural changes. The optimal range of alcohol presumably reduces protein aggregation by disrupting hydrophobic interactions that could seed the formation for an increased amount of protein aggregation. There are two alcohol levels that can affect the hGH: overall alcohol content of the solvent system and alcohol content that the hGH is exposed to upon mixing. The overall alcohol content of the combined solvents was held constant at 20/80 (v/v %) ethanol/water, the optimum levels determined previously (data not shown). Contact between hGH and high concentration ethanol was minimized by diluting the ethanol with water. Under the current process, the ethanol is diluted to 40 vol % and mixed with an equal amount of 100% aqueous hGH solution to create a final feed solution of 20 wt % ethanol. In order to determine if an even lower concentration of ethanol in the pre-mixed organic phase would have a beneficial effect on the chemical characterization of the final powder, the ethanol content of the organic phase was lowered to 30 vol % and then mixed with the aqueous hGH phase at a ratio of 2:1 organic:aqueous. There seems to be no advantage of exposing the hGH solution to a lower ethanol concentration than 40 vol %. Representative results for this example are set forth in Table 11.

TABLE 11

| | Water content | Aqueous: | | | VMGD | |
|---|---|---|---|---|---|---|
| | in organic | organic | HMWP | RE | 1 bar | FPF_{TD} < |
| Nozzle | phase (vol %) | ratio | (%) | (%) | (μm) | 3.3 μm |
| Single-hole | 60 | 1:1 | 1.6 | 95.4 | 6.1 | 70 |
| Single-hole | 70 | 2:1 | 1.6 | 95.9 | 6.5 | 68 |

### EXAMPLE 111

Batch/Static Mixing.

The spray drying solution can either be pre-mixed before spray drying or static mixed in-line immediately before entering the atomizer. A disadvantage to batch mixing is chemical degradation of the hGH when exposed to ammonium bicarbonate for prolonged periods. The advantage of static mixing is extended hGH stability over a course of days which allows prolonged spray-drying runs and improved productivity. Powders produced with both mixing methods but identical process conditions produced comparable powders with both batch and static mixing. Representative results for this example are set forth in Table 12.

TABLE 12

| | | HMWP | RE | VMGD | FPF_{TD} < | |
|---|---|---|---|---|---|---|
| Nozzle | Mixing | (%) | (%) | 1 bar (μm) | 3.3 μm | Method |
| Single-hole | Static | 1.1 | 96.7 | 7.4 | 70 | ACI-3, 60 1 pm |
| Single-hole | Batch | 1.6 | 96.1 | 7.7 | 64 | ACL-3, 60 1 pm |

### EXAMPLE 12

Process Conditions.

A. Spray Dryer Operating Pressure is regulated with an exhaust blower. All the work done in the laboratories in the Size 1 dryer has been done under slight vacuum (−2″ W.C). At commercial scale, the dryer is expected to operate under slight pressure.

B. Spray Dryer Outlet Temperature is the temperature at the outlet of the spray drying drum and is maintained by controlling the inlet temperature. As the outlet temperature increases the HMWP and the FPF increase and the moisture content decreases. The range of spray dryer outlet temperature for the single-hole nozzle was 35-70° C. and for the six-hole nozzle was 35-65° C. Representative results for this example are set forth in Table 13.

TABLE 13

| | | HMWP | RE | VMGD | FPF_{TD <} | |
|---|---|---|---|---|---|---|
| Nozzle | T_{out, sd} | (%) | (%) | 1 bar (μm) | 3.3 μm | Method |
| Single-hole | 40 | 1.5 | 97.2 | 7.1 | 57 | ACI-3, 28.3 1 pm |
| Single-hole | 60 | 2.1 | 96.3 | 6.6 | 65 | ACI-3, 28.3 1 pm |

C. Atomization Gas Rate is the rate of the high-velocity gas that creates the liquid droplets in two-fluid atomization. The mass gas to liquid ratio (atomization gas to liquid feed rate) is an important variable that affects mean droplet size. Increase in the ratio decreases droplet size, which may in turn increase FPF, although ratio values that are too high are not as effective. Thus, as atomization gas rate increases the VMGD tends to decrease as the FPF increases. The range of atomization gas rate for the single-hole nozzle was 38-120 g/min and for the six-hole nozzle was 50-120 g/min. Representative results for this example are set forth in Table 14.

TABLE 14

| | Atom gas | HMWP | RE | VMGD | FPF | |
|---|---|---|---|---|---|---|
| Nozzle | rate (g/min) | (%) | (%) | 1 bar (μm) | (%) | Method |
| Single-hole | 46 | 1.2 | 97.5 | 9.6 | 60 | ACI-2, 60 1 pm |
| Single-hole | 64 | 1.1 | 97.9 | 9.1 | 69 | ACI-2, 60 1 pm |
| Single-hole | 64 | 1.2 | 97.9 | 7.9 | 71 | ACI-2, 60 1 pm |
| Single-hole | 80 | 1.3 | 98.6 | 8.1 | 78 | ACI-2, 60 1 pm |
| Single-hole | 46 | 1.6 | 94.0 | 9.3 | 54 | ACI-3, 28.3 1 pm |
| Single-hole | 120 | 2.4 | 95.3 | 7.9 | 58 | ACI-3, 28.3 1 pm |

D. Liquid Feed Rate is the rate at which the liquid solutions are pumped into the atomizer and spray dryer. As the feed rates increase, the gas to liquid ratio decreases and thus the VMGD tends to increase as the FPF decreases. The range of liquid feed rates for the single-hole nozzle was 10-75 mL/min and for the six-hole nozzle was 10-40 mL/min. Representative results for this example are set forth in Table 15.

TABLE 15

| | Liquid feed rate | HMWP | RE | VMGD | FPF | |
|---|---|---|---|---|---|---|
| Nozzle | (mL/min) | (%) | (%) | 1 bar (μm) | (%) | Method |
| Single-hole | 15 | 2.2 | 97.3 | 7.5 | 77 | ACI-2, 60 1 pm |
| Single-hole | 50 | 1.8 | 96.6 | 8.4 | 66 | ACI-2, 60 1 pm |
| Six-hole | 25 | 3.4 | 97.4 | 10.2 | 66 | ACI-3, 60 1 pm |
| Six-hole | 40 | 3.0 | 97.3 | 15.1 | 43 | ACI-3, 60 1 pm |

E. Drying Gas Rate is the rate of the heating gas used to dry the droplets. The drying gas rate also controls the residence time within the dryer. The range of drying gas rate explored for the single-hole nozzle was 80-125 kg/hr. Representative results for this example are set forth in Table 16.

TABLE 16

| | Drying gas rate | HMWP | RE | VMGD | FPF | |
|---|---|---|---|---|---|---|
| Nozzle | (kg/hr) | (%) | (%) | 1 bar (μm) | (%) | Method |
| Single-hole | 80 | 1.7 | 97.9 | NA | NA | NA |
| Single-hole | 110 | 2.1 | 97.8 | NA | NA | NA |
| Single-hole | 110 | 2.8 | NA | 7.3 | 71 | ACI-2, 60 1 pm |
| Single-hole | 125 | 2.4 | NA | 8.0 | 70 | ACI-2, 60 1 pm |

What is claimed is:

1. A mass of biocompatible particles that consist essentially of, by weight of total hGH and sodium phosphate, about 80% to about 90% hGH and about 10% to about 20% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

2. The mass of claim 1, wherein the particles are in a receptacle and comprise from about 1.0 mg to about 25 mg of hGH.

3. The mass of claim 2, wherein the particles are in a receptacle and comprise from about 1.0 mg of hGH per receptacle.

4. The mass of claim 2, wherein the particles are in a receptacle and comprise from about 5 mg of hGH per receptacle.

5. The mass of claim 2, wherein the particles are in a receptacle and comprise from about 10 mg of hGH per receptacle.

6. The mass of claim 2, wherein the particles are in a receptacle and comprise from about 15 mg of hGH per receptacle.

7. The mass of claim 2, wherein the particles are in a receptacle and comprise from about 20 mg of hGH per receptacle.

8. The mass of claim 2, wherein the particles are in a receptacle and comprise from about 25 mg of hGH per receptacle.

9. The mass of claim 1, wherein the particles have a tap density less than about 0.3 g/cm³.

10. The mass of claim 1, wherein the particles have a tap density less than about 0.2 g/cm³.

11. The mass of claim 1, wherein the particles have a tap density less than about 0.1 g/cm³.

12. The mass of claim 1, wherein the particles have an aerodynamic diameter of from about 1 micrometer to about 3 micrometers.

13. The mass of claim 1, wherein the particles have an aerodynamic diameter of from about 3 micrometers to about 5 micrometers.

14. The mass of claim 1, wherein the particles further comprise DPPC.

15. The mass of claim 1, wherein the FPF <3.4 is greater than about 65%.

16. A mass of biocompatible particles that comprise, by weight of total hGH and sodium phosphate, about 90% to about 95% hGH and about 5% to about 10% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

17. A mass of biocompatible particles that comprise, by weight of total hGH and sodium phosphate, about 95% to about 100% hGH and about 0% to about 5% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

18. A mass of biocompatible particles that comprise, by weight of total hGH and sodium phosphate, about 93% hGH and about 7% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

19. A mass of biocompatible particles that comprise, by weight of total hGH and sodium phosphate, about 93.5% hGH and about 6.5% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

20. A mass of biocompatible particles that comprise hGH, DPPC and sodium phosphate, wherein the particles include, by weight of total hGH, DPPC and sodium phosphate, from about 75% to about about 90% hGH, and have a tap density less than about 0.4 g/cm³, a median geometric diameter of from between about 5 micrometers and about 10 micrometers, and an aerodynamic diameter of from about 1 micrometer to about 5 micrometers.

21. A mass of biocompatible particles that consist essentially of, by weight of total hGH, DPPC and sodium phosphate, about 80% hGH, about 14% DPPC and about 6% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

22. A method for treating a human patient in need of hGH comprising administering to the respiratory tract of a patient in need of treatment, in a single, breath actuated step, an effective amount of particles comprising, by weight of total hGH and sodium phosphate, about 80% to about 90% hGH and about 10% to about 20% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

23. The method of claim 22, wherein the particles have a tap density less than about 0.3 g/cm³.

24. The method of claim 22, wherein the particles have a tap density less than about 0.2 g/cm³.

25. The method of claim 22, wherein the particles have a tap density less than about 0.1 g/cm³.

26. The method of claim 22, wherein the particles have an aerodynamic diameter of from about 1 micrometers to about 3 micrometers.

27. The method of claim 22, wherein the particles have an aerodynamic diameter of from about 3 micrometers to about 5 micrometers.

28. The method of claim 22, wherein administering the particles pulmonarily includes delivery of the particles to the deep lung.

29. The method of claim 22, wherein administering the particles pulmonarily includes delivery of the particles to the central airways.

30. The method of claim 22, wherein administering the particles pulmonarily includes delivery of the particles to the upper airways.

31. The method of claim 22, wherein the particles further comprise DPPC.

32. The method of claim 22, wherein the FPF <3.4 is greater than about 65%.

33. A method for treating a human patient in need of hGH comprising administering to the respiratory tract of a patient in need of treatment, in a single, breath actuated step, an effective amount of particles comprising, by weight of total hGH and sodium phosphate, about 90% to about 95% hGH and about 5% to about 10% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

34. A method for treating a human patient in need of hGH comprising administering to the respiratory tract of a patient in need of treatment, in a single, breath actuated step, an effective amount of particles comprising, by weight of total hGH and sodium phosphate, about 95% to about 100% hGH and about 0% to about 5% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

35. A method for treating a human patient in need of hGH comprising administering to the respiratory tract of a patient in need of treatment, in a single, breath actuated step, an effective amount of particles comprising, by weight of total hGH and sodium phosphate, about 93% hGH and about 7% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

36. A method for treating a human patient in need of hGH comprising administering to the respiratory tract of a patient in need of treatment, in a single, breath actuated step, an effective amount of particles comprising, by weight of total hGH and sodium phosphate, about 93.5% hGH and about 6.5% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

37. A method for treating a human patient in need of hGH comprising administering to the respiratory tract of a patient in need of treatment, in a single, breath actuated step, an effective amount of particles comprising hGH, DPPC and sodium phosphate, wherein the particles include, by weight, from about 75% to about 90% hGH, and have a tap density less than about 0.4 g/cm³, a median geometric diameter of from between about 5 micrometers and about 10 micrometers, and an aerodynamic diameter of from about 1 micrometer to about 5 micrometers.

38. A method for treating a human patient in need of hGH comprising administering to the respiratory tract of a patient in need of treatment, in a single, breath actuated step, an effective amount of particles comprising, by weight of total hGH, DPPC and sodium phosphate, about 80% hGH, about 14% DPPC and about 6% sodium phosphate, wherein the particles have a tap density less than about 0.4 g/cm³, a median geometric diameter of from between about 5 micrometers and about 10 micrometers, and an aerodynamic diameter of from about 1 micrometer to about 5 micrometers.

39. A method of delivering an effective amount of hGH to the pulmonary system, comprising:
a) providing a mass of particles comprising, by weight of total hGH and sodium phosphate, about 90% to about 95% hGH and about 5% to about 10% sodium phosphate; and
b) administering, via simultaneous dispersion and inhalation, the particles, from a receptacle having the mass of the particles, to a human subject's respiratory tract, wherein the particles have a tap density less than about 0.4 g/cm³, a median geometric diameter of from between about 5 micrometers and about 10 micrometers, and an aerodynamic diameter of from about 1 micrometer to about 5 micrometers.

40. A method of delivering an effective amount of hGH to the pulmonary system, comprising:
a) providing a mass of particles comprising, by weight of total hGH and sodium phosphate, about 95% to about 100% hGH and about 0% to about 5% sodium phosphate; and
b) administering, via simultaneous dispersion and inhalation, the particles, from a receptacle having the mass of the particles, to a human subject's respiratory tract,
wherein the particles have a tap density less than about 0.4 g/cm³, a median geometric diameter of from between about 5 micrometers and about 10 micrometers, and an aerodynamic diameter of from about 1 micrometer to about 5 micrometers.

41. A method of delivering an effective amount of hGH to the pulmonary system, comprising:
a) providing a mass of particles comprising, by weight of total hGH and sodium phosphate, about 93% hGH and about 7% sodium phosphate; and
b) administering, via simultaneous dispersion and inhalation, the particles, from a receptacle having the mass of the particles, to a human subject's respiratory tract,
wherein the particles have a tap density less than about 0.4 g/cm³, a median geometric diameter of from between about 5 micrometers and about 10 micrometers, and an aerodynamic diameter of from about 1 micrometer to about 5 micrometers.

42. A method of delivering an effective amount of hGH to the pulmonary system, comprising:
a) providing a mass of particles comprising, by weight of total hGH and sodium phosphate, about 93.5% hGH and about 6.5% sodium phosphate; and
b) administering, via simultaneous dispersion and inhalation, the particles, from a receptacle having the mass of the particles, to a human subject's respiratory tract,
wherein the particles have a tap density less than about 0.4 g/cm³, a median geometric diameter of from between about 5 micrometers and about 10 micrometers, and an aerodynamic diameter of from about 1 micrometer to about 5 micrometers.

43. A method of delivering an effective amount of hGH to the pulmonary system, comprising:
a) providing a mass of particles comprising hGH, DPPC and sodium phosphate; and
b) administering, via simultaneous dispersion and inhalation, the particles, from a receptacle having the mass of the particles, to a human subject's respiratory tract,
wherein the particles include, by weight, from about 75% to about 90% hGH, and have a tap density less than about 0.4 g/cm³, a median geometric diameter of from between about 5 micrometers and about 10 micrometers, and an aerodynamic diameter of from about 1 micrometer to about 5 micrometers.

44. A method of delivering an effective amount of hGH to the pulmonary system, comprising:
a) providing a mass of particles comprising, by weight of total hGH, DPPC and sodium phosphate, about 80% hGH, about 14% DPPC and about 6% sodium phosphate; and
b) administering, via simultaneous dispersion and inhalation, the particles, from a receptacle having the mass of the particles, to a human subject's respiratory tract,
wherein the particles have a tap density less than about 0.4 g/cm³, a median geometric diameter of from between about 5 micrometers and about 10 micrometers, and an aerodynamic diameter of from about 1 micrometer to about 5 micrometers.
